(19) United States
(12) Patent Application Publication
(10) Pub. No.: US 2024/0200125 A1
(43) Pub. Date: Jun. 20, 2024

(54) ANALYSIS OF EFFLUENT

(71) Applicant: Droplet Biosciences, Inc., Cambridge, MA (US)

(72) Inventor: Wendy Winckler Adamovich, Melrose, MA (US)

(21) Appl. No.: 18/542,193

(22) Filed: Dec. 15, 2023

Related U.S. Application Data
(60) Provisional application No. 63/587,266, filed on Oct. 2, 2023.
(60) Provisional application No. 63/433,391, filed on Dec. 16, 2022.

(51) Int. Cl. C12Q 1/6851; C12Q 1/6876; G01N 33/68

(52) U.S. Cl. CPC C12Q 1/6851; C12Q 1/6876; G01N 33/6893; C12Q 2600/118; C12Q 2600/158; G01N 2800/26

(57) ABSTRACT

The invention provides methods of detecting, or predicting the aggressiveness of, a tumor by measuring cancer biomarkers in surgical drain fluid that may be collected from the site of a surgery such as a tumor resection or lymphadenectomy. Surgical drain fluid (SDF), and the lymph component of SDF in particular, contains biomarkers that demonstrably correlate to future cancer outcomes and that are thus predictive of the growth or aggressiveness of a tumor as well as surgical site infection and/or sepsis.

### TECHNICAL FIELD

The disclosure relates to early prediction of bacterial infection or sepsis in surgical drain fluid.

### BACKGROUND

Cancer is a leading cause of death globally. Early detection, while beneficial for most cancers, is often difficult. In part, this is because many cancers first develop without presenting any specific clinical symptoms, and diagnosis only occurs when the disease has reached a stage when it is difficult to treat.

Cancer detection has focused on cytology, imaging, and liquid biopsy in blood or plasma for the detection of cell-free tumor DNA. Blood is of high clinical interest because of its accessibility. Unfortunately, many of these methods lack sensitivity. As a result, early cancer detection, when tumor DNA is present as only a minute fraction of the DNA collected from blood or plasma, is often difficult. Moreover, due to the lack of sensitivity, progression of the disease and its response to therapeutic intervention are difficult to monitor.

Tissue, such as tumor tissue, generally is the most informative sample for diagnosis and prognosis of cancer. Unfortunately, tissue samples are often difficult to access and subject to limited availability, especially without performing a painful and invasive biopsy. In the context of cancer, often by the time tumors are detected, cancer has spread or progressed. Consequently, physicians and patients are often unable to make timely, informed decisions regarding therapeutic intervention.

One significant issue facing all patients undergoing surgery, including cancer patients receiving curative intent surgery, is the risk of surgical site infection. Post-surgical infections affect 2-4% of patients and are a leading cause of morbidity, mortality, and hospital readmission. Approximately 3% of patients who contract a surgical site infection will die.

Surgical drain fluid analysis to detect early infection is feasible in multiple surgical specialties and has the potential to address current unmet needs. Cardiothoracic surgeries have high rates of post-surgical infection, and chest tube placement to collect surgical drain fluid is standard-of-care for major surgeries. Orthopedic surgeries occur at very high volume and drains are placed electively for major surgeries. Post-surgical infection can necessitate the removal of devices, requiring additional surgeries and significant morbidity for the patient. Plastic surgery also has a moderate rate of infection and drains are common for some surgical procedures. Abdominal surgeries also have a high rate of infection but is particularly challenging for early sepsis identification as initial fluid often contains fecal bacteria that colonizes surgical drains. The collection of post-surgical lymphatic fluid using surgical drains represents a promising approach to detect infection before sepsis occurs in diverse surgical indications.

### SUMMARY

In a preferred embodiment, the invention provides methods for detecting a surgical site infection in lymphatic fluid from a surgical site by measuring a level of a microbial biomarker in the lymphatic fluid and then identifying a surgical site infection on the basis of the measured biomarker level. As set forth herein, numerous different biomarkers or combinations of biomarkers are useful in the invention. In addition, any measuring technique known in the art is useful for identifying, quantifying, and assessing biomarkers indicative of surgical site infection.

In another aspect, the invention provides methods for the prediction of sepsis based on biomarkers identified, quantified, or assessed in surgical drain fluid or lymphatic fluid.

In addition to the foregoing, the invention provides methods of detecting and/or predicting the aggressiveness of a tumor by measuring cancer biomarkers in lymphatic drain fluid produced in proximity to a tumor. In particular, methods of the invention involve measuring a biomarker, such a protein or nucleic acid biomarker, in lymphatic fluid, in surgical drain fluid for the prediction of recurrence, disease severity, and/or treatment options. Effluent or drain fluid in proximity to a tumor has a characteristic composition that may change over time but typically includes blood or plasma, lymph and interstitial fluid, and any wash fluid used in a medical procedure. Conventionally, those fluids are discarded as waste. In fact, some patients are sent home with implanted surgical site drains and instructions on how to clear and discard collected fluid. The present invention makes use of the insight that such surgical drain fluid (SDF), and the lymph component of SDF in particular, contains biomarkers that demonstrably correlate to future cancer outcomes such as recurrence; and that are predictive of the growth or aggressiveness of cancer. The SDF is also useful to inform therapeutic selection and interventional outcomes.

Drain fluid may be obtained from medical procedures such as surgeries, biopsies, catheterizations, and the like. Drain fluid collected from medical procedures is a rich and more reliable source of biomarkers indicative of disease as compared to plasma. Plasma has been explored for measurement of tumor-derived cell-free DNA (ctDNA) as well as DNA associated with circulating tumor cells (CTCs) as biomarkers for susceptibility to cancer as well as recurrence. However, the detection of such biomarkers in plasma is relatively non-specific. Moreover, the concentration of analytes in plasma sample is relatively low due to the dilution of analytes within a patient's plasma. Obtaining SDF proximal to a tumor provides a better insight into the disease such as metastasis, recurrence, as well as stage of the disease since it is collected from the tumor micro-environment (TME).

In one feature, methods of the invention are useful for prediction of recurrence, metastasis, or the aggressiveness of a tumor or for the detection of recurrence or minimal residual disease (MRD). The invention makes use of the fact that cancer-cells, as well as the tissue microenvironment surrounding a tumor, and the repertoire of immune cells characteristically present in response to a tumor shed a variety of biomarkers that are measurable in lymphatic fluid. Lymphatic fluid, e.g., as is found in surgical drain fluid, reliably includes predictive biomarkers indicative of tumor aggressiveness, recurrence, staging and therapeutic outcome.

Measuring those biomarkers—cells, nucleic acids, proteins, or others—is informative of the presence and aggressiveness of a tumor. Specifically, measured levels of certain biomarkers in drain fluid are used to predict the recurrence of a cancer after treatment, the survival of patients after treatment, or imminent or future metastasis. Biomarkers measured according to the invention can reveal MRD earlier than can be discovered by conventional techniques. Additionally, biomarker measurements using methods of the invention, e.g., in lymphatic fluid, are also predictive of sepsis, or surgical site infection (SSI), at a time before conventional methods can predict the onset of those conditions.

In certain aspects, the invention provides methods of predicting cancer recurrence. Such methods include obtaining lymphatic fluid from a site proximal to a tumor, measuring a level of a predetermined protein in the lymphatic fluid, and predicting recurrence of the cancer on the basis of the measured level. The measured protein may be an inflammatory protein, such as a cytokine. For example, the protein may be an interleukin, e.g., IL-1β or IL-6. The lymphatic fluid may be obtained from site of surgical intervention. In some embodiments, the lymphatic fluid is obtained as fluid that drains from the surgical site and is obtained during and/or after surgical removal intervention. Preferred methods may include making first and subsequent measurements, i.e., during and/or after the surgery, to detecting a change or a trend in a level of a biomarker. Specifically, methods of the invention utilize the velocity of change as a predictor of disease outcome and/or progression. Optionally, the method includes measuring levels of a plurality of proteins such as cytokines in the lymphatic fluid.

Proteins are important biomarkers of disease, drug response, and the likelihood of disease recurrence. In one aspect, the invention provides methods to discover novel protein targets and biomarkers in SDF and plasma. The invention also provides methods to investigate the differences in the proteome of SDF (lymphatic fluid/lymph) and plasma to provide increased sensitivity and specificity of diagnosis, progression, therapeutic selection, and therapeutic efficacy.

The inventors compared the protein profiles of lymph (surgical drain fluid proximal to the tumor) and plasma from 44 Human papillomavirus (HPV) negative head and neck cancer patients. The assay provides matched pairs of antibodies, each antibody labelled with unique oligonucleotide-barcodes per target protein. When matched antibodies bind to the same target protein in solution, the oligonucleotide-barcode strands on each antibody hybridize. The barcodes are then amplified, and the resulting amplicons are read using known techniques in the art such as qPCR or NGS. The assay results show highly differentiated protein repertoires for lymph and plasma. Further, there are many more protein biomarkers that predict recurrence in lymph than in plasma, and nearly all do not overlap, indicating opportunity for unique biomarker discovery in lymph fluid. The protein biomarkers in the lymph include canonical TME biomarkers as well as novel TME regulators. The terms “lymph” and “surgical drain fluid” or “drain fluid” are used interchangeably herein.

In certain embodiments for the detection of tumor mutational burden, circulating tumor DNA (ctDNA) from the lymphatic fluid is sequenced to identify a number of tumor-related mutations.

In preferred embodiments, the measuring step includes performing an assay to quantify the level of the protein the lymphatic fluid. For example, the assay may include binding labeled antibodies to the proteins and detected labels in an assay, such as an ELISA sandwich assay. The measured protein may be IL-1β and the method may include predicting recurrence of the tumor when the IL-1β is present at a concentration of at least 30 pg/mL in the lymphatic fluid.

Aspects of the invention provide methods of therapeutic selection. Such methods include obtaining lymphatic fluid produced proximal to a tumor, measuring a level of a predetermined protein or nucleic acid biomarker in the lymphatic fluid, and selecting a treatment on the basis of the measured level(s). Preferably, the lymphatic fluid is obtained after a surgery to remove the tumor, and the treatment is selected for adjuvant therapy. The protein biomarker may be a pro-inflammatory cytokine. The measured level may be used to select an adjuvant therapy. The selected treatment may include an antibody such as an interleukin inhibitor (e.g., the IL-1beta inhibitor canakinumab). Optionally the selected treatment also includes the interleukin inhibitor in combination with a treatment such as chemotherapy, radiation, or an immunotherapy (e.g., a checkpoint inhibitor). In one exemplary embodiment, the measured protein is IL-1beta and the selected treatment comprises an IL-1β inhibitor.

Other aspects of the invention provide methods for predicting therapeutic outcome. Such methods include obtaining lymphatic fluid produced proximal to a tumor in a subject who has been treated for cancer by a neoadjuvant therapy and surgery, measuring a level of a biomarker in the lymphatic fluid, and determining a pharmacodynamic effect of the neoadjuvant therapy on the basis of the measured level. The measured protein may be, for example, a cytokine, a growth factor, or an interleukin, such as IL-1β or IL-6. The neoadjuvant therapy may include, for example, an IL-1β inhibitor. The neoadjuvant therapy may also include the IL-1β inhibitor in combination with a treatment, such as chemotherapy, radiation, or an immunotherapy (e.g., a checkpoint inhibitor). In certain embodiments, the fluid is obtained at the site of tumor resection. The lymphatic fluid may be obtained as drain fluid that drains from the surgical site and optionally may be obtained both during and after (e.g., two or more different times) surgical removal of the tumor, wherein protein levels are measured at both times.

In related aspects, the invention provides methods for adjuvant therapy. Those methods include obtaining lymphatic fluid produced proximal to a tumor in a patient who has been treated for cancer, measuring a level of a predetermined protein in the lymphatic fluid, and treating the patient with an IL-1β inhibitor when the measured level of the predetermined protein in the lymphatic protein is at least a predetermined level. The protein is preferably a cytokine or interleukin, such as IL-1β. The methods may include treating the patient with the IL-1β inhibitor when the measured level of IL-1β in the lymphatic fluid is at least about 30 pg/mL. Preferably, the lymphatic fluid is collected as fluid that drains from a surgical site, such as the site of tumor resection. The fluid may be obtained during the surgical removal of the tumor, after, or both. Two or more measurements may be made at different times to detect a trend in changing levels of the protein in the lymphatic fluid. The methods may also include treating the patient with the IL-1β inhibitor in combination with another treatment such as chemotherapy, radiation, or immunotherapy (e.g., a checkpoint inhibitor).

In certain aspects, the invention provides tumor analysis methods. Those methods include obtaining lymphatic fluid from a subject, measuring levels of a plurality of biomarkers in the lymphatic fluid, and associating the measured levels of biomarkers in the fluid with the presence or aggressiveness or metastatic potential of a tumor in the subject. The prediction may include cancer recurrence, metastasis, or progression. Preferably, the fluid is obtained from a site of, or proximal to, the tumor. For example, during and/or after tumor resection, the drain fluid is collected from the surgical site of the tumor removal.

The measuring step may include sequencing nucleic acid from the lymphatic fluid to obtain sequence data and detecting tumor mutations in the sequence data. The biomarkers may include mutations specific to a tumor, such that higher numbers of tumor mutations predict a poor probability of survival. In some embodiments, the lymphatic fluid comprises tumor nucleic acid, and the method includes reporting the presence of minimal residual disease when tumor mutations are detected in the fluid, i.e., when the measuring reveals a threshold mutation count.

In some embodiments, the measuring step includes sequencing nucleic acid to identify T cell receptors (TCR) present in the sample, and to create a profile of a TCR repertoire. For example, the measuring step may include performing multiplex amplification from gDNA or RNA from T-cells from the fluid to produce amplicons and sequencing the amplicons to determine a T-cell receptor (TCR) repertoire. The methods may include providing a report with a measure of clonality of the tumor based on T-cell diversity measured by TCR sequencing. The methods may include predicting a tumor to be more aggressive, with higher probability of metastasis, when TCR sequencing reveals a low diversity index, i.e., a population of cells indicating near monoclonality. With TCR sequencing, methods may include correlating TCR diversity measured from lymph in lymphatic fluid to TCR diversity measured from tissue and predicting that the patient will be non-responsive to an immunotherapy when tissue- and lymph-derived TCR diversity are highly correlated. Preferably the lymphatic fluid is surgical drain fluid (SDF), the measuring step includes TCR sequencing from the SDF, and the method includes reporting a measure of clonality of the tumor based on the TCR sequencing.

In certain embodiments, the measuring step includes performing an assay to detect a plurality of pre-determined proteins. Such an assay may include binding labeled antibodies to the proteins. The method may include detecting or sequencing proteins or nucleic acids from tumor-associated T cells in the drain fluid and providing a report with a profile of an immune microenvironment of the tumor based on the proteins or nucleic acids. Biomarkers may include proteins and may specifically include one or more cytokine, chemokine, or growth factor. Preferred proteins may include a plurality of pre-determined proteins, such as GM-CSF; VE-cadherin; BDNF; cathepsin D; CEA (CEACAM-5); EGF; EGFR (ErbB1); eotaxin (CCL11); FGF-2; GRO alpha (CXCL1); haptoglobin; HGF; HGFR (c-Met); IFN alpha; IFN gamma; IGFBP-2; IGFBP-3; IL-1 alpha; IL-1 beta; IL-10; IL-12p70; IL-13; IL-15; IL-17A (CTLA-8); IL-18; IL-IRA; IL-2; IL-21; IL-22; IL-23; IL-27; IL-31; IL-4; IL-5; IL-6; IL-7; IL-8; IL-9; IP-10 (CXCL10); MCP-1 (CCL2); MIA; MIP-1 alpha (CCL3); MIP-1 beta (CCL4); MIP-4 (CCL18); NGF beta; beta-2-microglobulin (B2M); PDGF-BB; periostin (OSF-2); PIGF-1; RANTES (CCL5); SCF; SDF-1 alpha; TNF alpha; TNF beta; VEGF-A; and VEGF-D. Preferred embodiments measure the concentration of at least one, preferably two, or three or more of IGFBP-2, B2M, FGF-2, IL-1 beta, IL-6, and IL-7 in lymph or drain fluid (e.g., present as proteins in pg/mL) and predicting metastasis or recurrence when one or more of those proteins is present in at least a threshold concentration.

In certain embodiments, the biomarkers include haptoglobin, IGFBP-2, and B2M, and high levels of biomarkers in the drain fluid predicts metastasis or recurrence after treatment.

In related embodiments, the biomarkers include at least one of FGF-2, IL-1 beta, IL-6, and IL-7, and high levels of biomarkers in the drain fluid predicts significant progression of the tumor or recurrence of the tumor after treatment.

Preferably, the obtaining step is performed after a patient has been treated to eradicate the tumor (e.g., tumor resection), and the measuring step includes measuring IL-1 beta in the drain fluid, and the method include predicting recurrence of the tumor when the IL-1 beta is present in an amount of at least 30 pg/mL in the drain fluid.

The lymphatic fluid may be surgical drain fluid that collects at a surgical site. For purposes of the present disclosure, lymphatic fluid and drain fluid are synonymous. Surgical drain fluid may be collected into a collection bulb or vessel. Preferably the drain fluid comprises lymph. The drain fluid may be obtained via a drain that includes a tube positioned to collect the drain fluid from a surgical site where a lymph node has been removed.

In certain microbial detection, or sepsis, embodiments, the method includes measuring a level of a microbial biomarker in the drain fluid. For example, the measuring may include sequencing microbial ribosomal nucleic acid and identifying a genus of a bacterium. Optionally, the microbial biomarker is measured at two different times to detect an increasing level of a microbe in the patient. The method may include reporting or predicting sepsis in the patient when the increasing level of the microbe is present, e.g., particularly when a genus of the microbe is Staphylococcus, Clostridium, Flaviobacterium, Neisseria, Pseudomonas, or Sphingomonas.

### BRIEF DESCRIPTION OF THE DRAWINGS

FIG. 1 diagrams a tumor analysis method.

FIG. 2 shows a workflow for analyzing nucleic acids in SDF.

FIG. 3 shows mutations detected by sequencing DNA from lymph versus plasma.

FIG. 4 shows that lymph has higher tumor fraction than plasma.

FIG. 5 plots survival using count of mutations in SDF.

FIG. 6 shows high TCR overlap for lymph and tumor tissue samples. This indicates that lymph has a high representation of tumor-infiltrating lymphocytes.

FIG. 7 illustrates multiplex protein quantification.

FIG. 8 is a heat map of proteins from a metastasis panel.

FIG. 9 shows concentration of IL-1 in drain fluid for patients with known outcomes.

FIG. 10 shows that FGF-2 in drain fluid is significantly predictive of recurrence.

FIG. 11 shows that IL-7 in drain fluid is significantly predictive of recurrence.

FIG. 12 shows that IL-1 beta, FGF-2 & IL-7 effects are robust across dilutions.

FIG. 13 shows that IL-1β stratifies recurrence.

FIG. 14 is a graph showing relative abundances of plurality of bacteria in drain fluid.

FIG. 15 shows that DNA concentrations in lymph in SDF is higher than in plasma.

FIG. 16 shows fragmentation patterns of cfDNA found in lymph.

FIG. 17 shows that eotaxin correlates to progression.

FIG. 18 shows that GRO alpha (CXCL1) correlates to progression.

FIG. 19 shows that IL-1RA correlates to progression.

FIG. 20 shows that IL-2 correlates to progression.

FIG. 21 shows MIP-1 beta (CCL4) correlated to progression.

FIG. 22 shows a K-M plot of recurrence with DNA and protein in drain fluid.

FIG. 23 Shows IL-1β stratification of recurrence in ELISA and Luminex assays.

FIG. 24 shows that IL6 tracks IL-1β impact on disease progression.

FIG. 25 shows lymph and plasma protein repertoires are distinct.

FIG. 26 shows lymph is enriched for multiple TME-associated proteins in recurred patients that are absent in plasma.

### DETAILED DESCRIPTION

Lymphatic fluid contains tumor biomarkers that can be interrogated to detect and predict the recurrence, presence, and/or aggressiveness of a tumor. Lymphatic fluid is included in fluid that drains or is irrigated or removed from a surgical site. For example, a tumor resection or a lymphadenectomy (or lymph node dissection) may be performed. Certain types of cancer have a tendency to produce lymph node metastasis, a phenomenon particular characteristic of melanoma, head and neck cancer, differentiated thyroid cancer, breast cancer, lung cancer, gastric cancer and colorectal cancer. For lymph node dissection, an incision is made in the skin near the affected lymph nodes. The lymph nodes and typically nearby lymphatic tissue and underlying soft tissue and removed. During, and after, such a surgical removal, fluid drains from the surgical site. For a tumor resection, an incision is made, and the tumor is surgically removed. In both examples, fluid drains from the surgical site and that fluid may be referred to as drain fluid. The composition of that drain fluid may vary over time (e.g., may include saline during a surgery when saline is used to irrigate and wash the site), but the drain fluid will reliably include lymph or lymphatic fluid, typically along with blood and interstitial fluid.

The fluid that drains from the site of such a surgery may include both fluids originating in the patient and any wash used to irrigate the site, such as sterile saline. The surgical drain fluid may typically include different contributing materials including, for example, blood and lymph. However, compared to a venous blood draw, that fluid will include a rich amount of lymphatic fluid. Also, due to the relationship between the surgery and its purpose, fluid that drains from the surgical site has the potential to be rich in material that is specific to the anatomical target of the surgery and the surrounding tissue. For example, when a tumor resection is performed to remove a tumor, fluid that drains from the site from which the tumor was removed may be rich in material from the proximal tumor or its tumor microenvironment.

Here, the invention uses lymphatic fluid and biomarkers found within the fluid to detect the continued presence of the tumor and/or to predict recurrence or aggressiveness of the tumor. A particular insight of the invention is that lymph contains T-cells and biomarkers of T-cells and, when surgical drain fluid is collected from the site of a surgery such as a tumor resection or lymphadenectomy proximal to a tumor, the T-cells, products thereof, and other tumor biomarkers in the drain fluid are characteristic of, and predictive of, the aggressiveness and continuing presence of the tumor.

Methods of the invention may include measuring any suitable biomarkers in lymphatic fluid. For example, in various embodiments, the fluid is used as a source of T-cell or tumor DNA that is sequenced for mutations detection and counting. Mutation counts in lymphatic fluid characterize the presence and state of the tumor in a patient. e.g., counting tumor mutations in SDF is predictive of tumor aggressiveness. In another example, T-cell sequencing is performed to profile the T-cell receptor (TCR) repertoire present among T-cells in the fluid. The TCR repertoire can be used to evaluate clonality (e.g., by calculating a diversity index for TCRs profiled by sequencing T-cell gDNA in drain fluid). Methods of the invention also profile proteins present in lymphatic fluid. Results shown below demonstrate that certain proteins are good biomarkers for future tumor aggressiveness and correlate to residual disease and future recurrence. Another example included herein is the detection of surgical site infection prior to the onset of sepsis from analysis of lymphatic fluid. Microbial biomarkers are also measured (e.g., by sequencing to detect and classify microbial ribosomal segments). In an example, microbial 16S markers and—in particular—microbial 16S markers that increase quantitatively over at least two time points over time for certain sepsis-implicated pathogenic bacteria are effective as a very early predictors of sepsis, useful long before clinical blood cultures can give a reliable result.

Thus, the invention provides methods of characterizing SDF or lymph for biomarkers predictive of tumor aggressiveness and future sepsis.

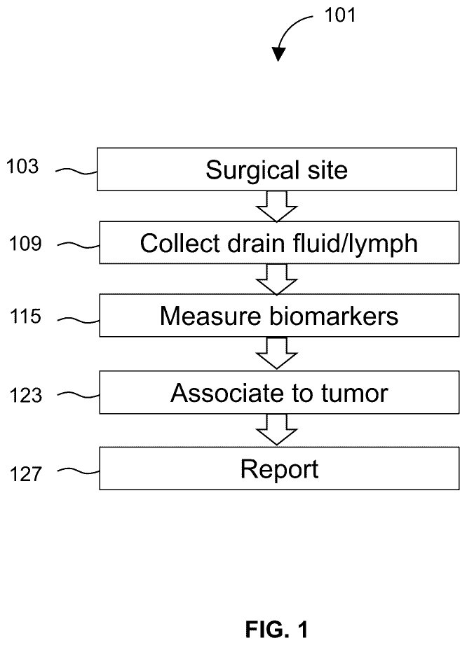

FIG. 1 diagrams a tumor analysis method 101. The method is primarily directed towards addressing 103 a site of a surgery such as a lymphadenectomy or tumor resection. The method includes obtaining 109 drain fluid or lymph from a subject, i.e., from the surgical site. The fluid may be collected at any suitable time. For example, noting that the surgical incision must be made prior to accomplishing the primary purpose of the surgery, fluid may be collected 109 as or once the incision is made, before other stages of the surgery progress. The collection 109 may be of fluid that simply drains naturally, or the site may be irrigated. The fluid may be collected during the progression of the surgery. For example, during dissection of a lymph node, fluid that is collected may be particularly rich in biomarkers of the lymph node microenvironment or the tumor microenvironment. Fluid may be collected immediately after the surgery, e.g., within hours or days before the incision has healed. In other embodiments, it may be that SDF collected after surgery, e.g., days, weeks or months after the surgery may be useful to detect or predict minimal residual disease or recurrence. In preferred embodiments, lymphatic fluid, such as is present in drain fluid, is collected more than once, and a level of a biomarker (such as a cytokine) in the lymphatic fluid is measured for the two different collection times, to establish a trendline indicating static, increasing, or decreasing levels of the biomarker in the lymphatic fluid.

The method 101 includes measuring 115 levels of a plurality of biomarkers in the drain fluid. As shown in greater detail below, the measurement 115 may include sequencing nucleic acid from the drain fluid or lymph. Nucleic acid such as gDNA may be sequenced and the sequences may be analyzed (e.g., compared to a reference such as a human genome, to “matched normal” sequences, or to matched tumor sequences) to detect and count mutations. Nucleic acid may be subject to TCR sequencing, e.g., to detect the presence of T cells and measure TCR clonality or diversity in the fluid. The fluid may be subject to an assay such as an ELISA to detect or quantify certain predetermined proteins present in the drain fluid. In but one example, the concentration of certain proteins (e.g., IL-1 beta, FGF-2, IL-6, and IL-7) in lymph or drain fluid correlates to future recurrence of cancer. Any such protein may be measured to predict recurrence. Additionally, bacterial biomarkers (such as 16S gene sequences) may be detected and measured.

An exemplary method 101 includes associating 123 the measured levels of biomarkers in the drain fluid with the presence or metastatic potential of a tumor in the subject or to a clinical condition such as sepsis. The lymphatic system regulates immune responses to pathogens and cancer and is characterized by a circulating fluid, lymphatic fluid or lymph, that circulates through the lymphatic system separately from the bloodstream. Lymph is a proximal source of lymphocytes, proteins, and other biomarkers. Methods of the invention involve collecting 109 and stabilizing lymph from surgical drain fluid, which is then useful as a novel analyte for multi-omic analysis. Drain fluid is distinct from blood. Drain fluid typically includes blood (sanguineous or serosanguinous fluids) but also interstitial and lymphatic fluid. Methods of the invention make use of a comprehensive mutli-omic characterization of the SDF and lymph. The invention makes use of the insight that tumor DNA can be detected in SDF. For example, in HPV+ head and neck cancer, the presence of tumor DNA in lymph or drain fluid is associated with, and a marker of, recurrence. Such predictors may also be available in lymph and drain fluid for other cancers such as, for example, lung and bladder cancer.

Measuring biomarkers in lymph or drain fluid may include looking at or evaluating one or any combination of aspects of nucleic acids, proteins, cells, and microorganisms. For example, DNA can be interrogated for yield, fragmentation patterns, mutations, and receptor diversity. Protein concentration or presence or abundance of certain pre-determined proteins such as immune proteins in lymph or drain fluid may be measured. Proteins in such samples may serve as early signals or predictors of recurrence or metastasis. For example, the concentration of a cytokine, such as IL-1β, in fluid that drains from a surgical site, is predictive of recurrence of the tumor. Additionally, lymph or drain fluid may be evaluated for its content of cells or the products of certain cells such as T-cells, or the overlap with tumor-derived T-cells. Finally, in surgical site infection and sepsis embodiments, lymph or drain fluid is evaluated for markers of the presence of a spectrum of pathogenic bacteria.

The characterization of such markers in lymph or drain fluid has a variety of potential applications include being useful for the detection of minimal residual disease (MRD) after treatment, therapeutic selection, prediction of therapeutic efficacy including for specific immune-oncology therapeutics using tumor-associated lymphocytes, and/or as a test for the prediction of surgical site infection (SSI), which itself (the SSI test) may be standalone or in connection with an MRD test. Any marker may be assayed in a multi-omic probe of lymph or drain fluid including, for example, nucleic acid, proteins, cells, or microbial markers. Certain embodiments include sequencing nucleic acid extracted from lymph or surgical drain fluid (SDF).

### Recurrence Prediction

FIG. 1 gives the steps of a method 101 that is useful to (i) predict recurrence of a tumor, (ii) evaluate success of a neoadjuvant therapy, or (iii) to identify patients that will benefit from certain adjuvant therapies all in the interest of predicting and/or avoiding recurrence. The method 101 includes addressing 103 a site of a surgery such as the surgical site from which a tumor is being, or has been (or both), removed. The disclosure includes the insight that fluid will drain from such a site. Traditionally, that fluid was discarded as waste and, in some cases, even washed away (e.g., with a saline) on a theory of keeping the site clean and washing away the waste. Here, instead of washing away and discarding the surgical drain fluid, the drain fluid, and the lymphatic component of that surgical drain fluid, is collected 109. The method 101 includes measuring 115 levels of a biomarker in the drain fluid.

Preferably, the drain fluid is subject to an assay to measure a concentration of a protein, such as a cytokine. For example, it has been found that certain interleukins such as IL-1β are predictive of recurrence after surgery to remove a tumor. Or, similarly, are markers of effectiveness of neoadjuvant therapy (no or low IL-1β indicates that the neoadjuvant therapy had its intended clinical effect) or aid in selection of adjuvant therapy strategies (e.g., high concentration of IL-1β calls for certain aggressive adjuvant therapy (IL-1 inhibitor) or combination of therapies such as an IL-1β inhibitor with chemo or radiation or immunotherapy. The method 101 includes associating 123 the measured level of the protein to a prediction for the tumor. For example, IL-1β present at 30 pg/mL or above after surgical removal of the tumor is associated with a high chance of tumor recurrence. Based on the association between the measured level of the biomarker (e.g., [IL-1β]), the method 101 includes providing 127 a report with information about a probability of tumor recurrence or a success of a therapy or a recommendation for a further therapy.

In recurrence prediction, the report may be as simple as identifying certain patients with a high probability of recurrence of the tumor after surgery. More specifically, the report may identify patients with biological indicia of a need for certain adjuvant therapies (e.g., an IL-1β inhibitor when [IL-1β] in lymphatic fluid of drain fluid exceeds threshold levels).

### Mutation Detection Counting

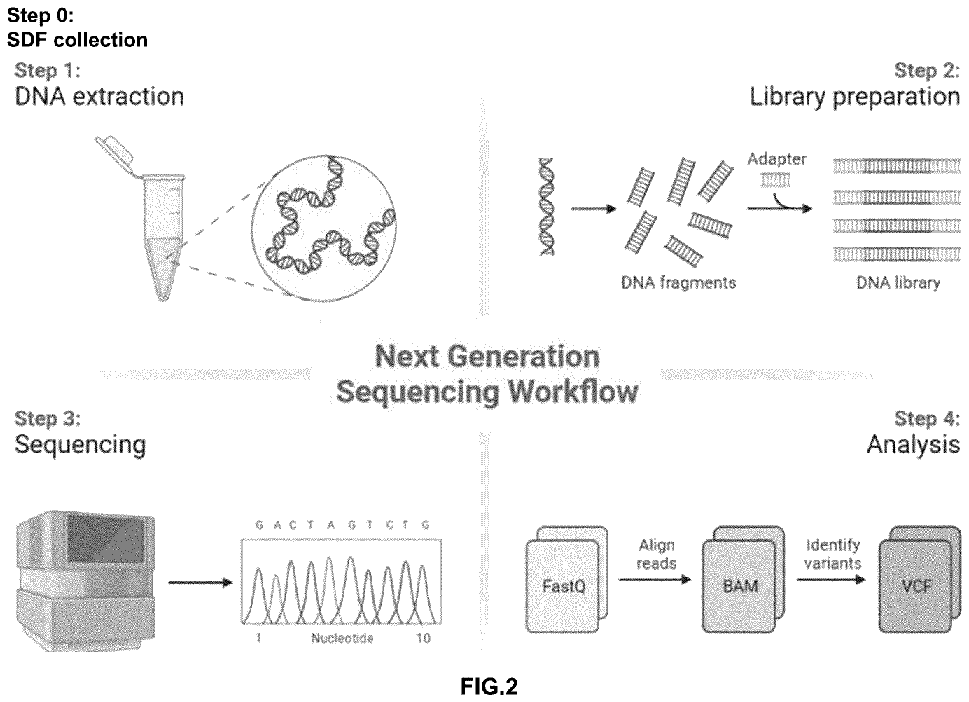

FIG. 2 shows a workflow for analyzing nucleic acids in SDF, in particular to determine detectable mutation count. Embodiments were performed using 22 HPV− head and neck cancer patients. Initially (at step 0), SDF was collected 109. DNA is extracted and, at step 2, a sequencing library is prepared. Any suitable library preparation may be used. For example, extracted DNA can be fragmented (e.g., using a sonicator) and ligated to adaptors. The fragments are amplified from the adaptors to yield amplicons that are sequenced at step 3.

Sequencing may be performed on any suitable platform including, for example, Sanger sequencing, so-called Next Generation Sequencing (NGS) technologies such as those instruments and methods offered by ILLUMINA, ROCHE, or ULTIMA, single molecule sequencing using instruments or technologies offered by PACBIO or OXFORD NANOPORE, others, or combinations thereof. The sequencing may target specific genetic segments, genes, mutations, or panels of mutations. For example, the method was performed on 22 patients with the TRUSIGHT Oncology 500 panel from Illumina, at 5000× coverage for lymph and plasma and at 200× coverage for tumor and normal.

Common sequencing instruments yield sequence reads, e.g., in the FASTA or FASTQ format. The reads may be aligned to a reference, such as the “hg37” human genome reference, and the alignments can be reported as, and saved as, a sequence alignment map (SAM) or binary alignment map (BAM) file. From the comparison to the reference genome, places where the sequence reads do not match (vary from) the reference may be “called” as variants (aka mutations), which is sometimes reported and stored in a variant call format (*.vcf) file, aka a VCF file. Such method steps may proceed with steps and formats as described in U.S. Pat. No. 8,209,130, incorporated by reference. Mutations (or variants) may be read or counted from the VCF files.

The results from the TRUSIGHT sequencing provided a count of mutations (from among 500 loci implicated in cancer), which may be referred to as mutation count. An initial goal of the study was to benchmark mutation detection in lymph compared to plasma. The study was used to assess recurrence prediction from lymph using point mutations. At step 0, SDF was collected. As discussed, SDF is preferably collected as the drainage from a surgical site. In preferred embodiments, the drain fluid is obtained from a site proximal to the tumor. For example, during or after a lymphadenectomy, the site of lymph node dissection is often at the closest lymph node to a tumor (e.g., that has been detected by, e.g., x-ray or other means). The SDF may include that fluid that collects in the body of the subject at a site of surgery. The SDF may be collected into a collection bulb or vessel. While the compositions of drain fluid may change over time during and after the surgery, typically it will always contain lymph (very early, e.g., during the incision, it may be predominantly blood). Starting at the time of surgery, it may be found that the amount of lymph present increases as the surgical site heals. The SDF may be obtained via a drain (such as a JP drain) that includes a tube positioned to collect the drain fluid from a surgical site where a lymph node has been removed.

As shown, the measuring 115 step may include sequencing nucleic acid from the drain fluid to obtain sequence data and detecting tumor mutations in the sequence data. Results from the study show that lymph outperforms plasma when using count of mutations or mutant allele fraction (MAF) to predict MRD after treatment of a head and neck cancer. Outcomes of study patients were known. Specifically, 12 recurred, 10 non-recurred w/1+ year follow up.

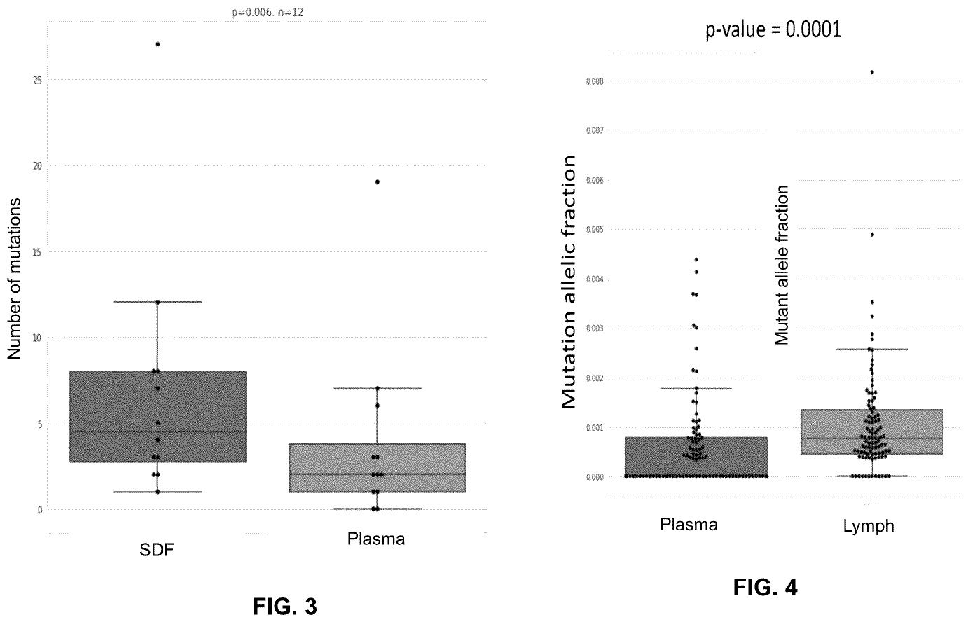

FIG. 3 shows that more mutations were detected by sequencing DNA from lymph that from plasma. An application of the method is when mutation count for a tumor is known prior to a tumor resection surgery. The pre-surgical mutation count may be stored, e.g., in a file. The tumor resection may be performed, and then SDF collected later. Nucleic acid from the SDF may be sequenced to determine mutation count. If the majority of the tumor mutations are observed in SDF, the method predicts recurrence. If the SDF TMB<<pre-surgical TMB, then the method predicts a low probability of recurrence.

FIG. 4 shows that lymph has higher tumor fraction (MAF) than plasma. This indicates that there is more signal for MRD detection in SDF than in plasma, yielding greater diagnostic sensitivity.

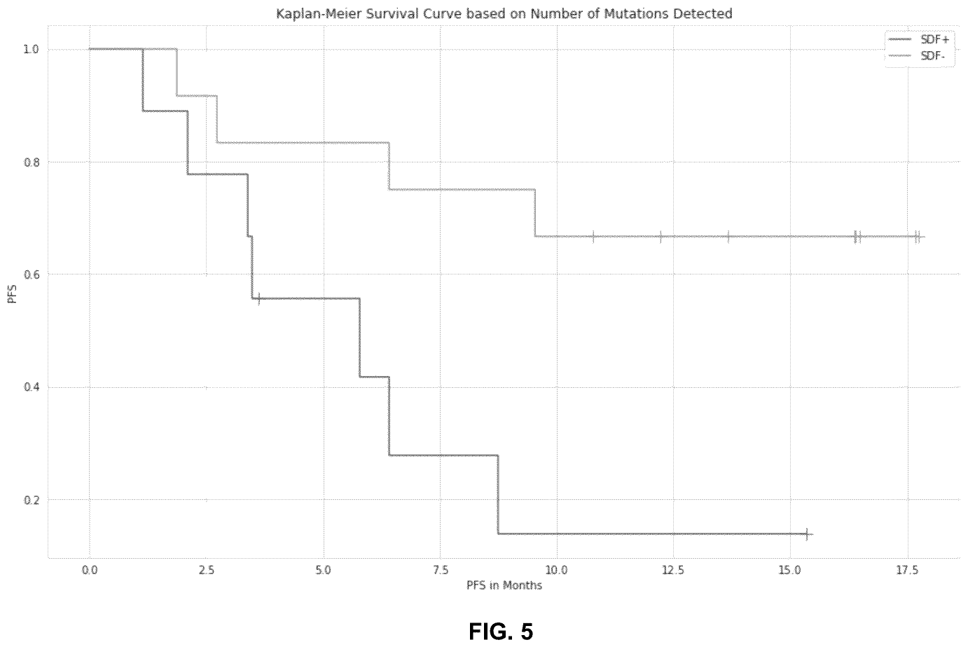

FIG. 5 plots progression-free survival over months after surgery for patients with a threshold count of mutations in SDF (SDF+) versus those without (SDF−). That is, the figure shows that sequencing nucleic acid from lymph or drain fluid to detect and count mutations implicated in cancer is useful to predict recurrence of the cancer. As can be seen, the two populations quickly diverge and, over time, the divergence only grows. The graph includes results from 21 patients with HPV− head and neck cancer from lymph collected via SDF 24 hours after surgery (p=0.02).

In such embodiments, the drain fluid includes lymph that includes tumor nucleic acid, and the method 101 may include reporting 127 a probability of survival or the presence of minimal residual disease (MRD) when (e.g., a count of) tumor mutations are detected in the lymph. In such embodiments, the measuring step 115 may include sequencing nucleic acid to produce sequence reads (FASTQ file) and comparing the reads to a reference to identify and count mutations (BAM and VCF files).

### T-Cell Receptor Repertoire.

Other embodiments include sequencing analyses to measure the representation of T-cells in lymph to identify T-cell clones shared between lymph and tumor tissue samples. Demonstrating that T cell clones may be found both in lymph or drain fluid as well as in tissue samples from tumors or the tumor microenvironment demonstrates that lymph or drain fluid is useful to profile a T-Cell Receptor (TCR) repertoire of a tumor. In such embodiments, the measuring step 115 of the method includes detecting and sequencing T cell DNA in the drain fluid. This was performed over eight patients with HPV− head and neck cancer. The sequencing proceeded by the paired, parallel sequencing of lymph gDNA and tumor DNA. The subject population included recurred, non-recurred, and Pembrolizumab neoadjuvant treated patients.

This was performed to assess lymph as a non-invasive window into T cell representations and identify clones shared between lymph and tumor tissue samples. T-Cell Receptor (TCR) sequencing was performed substantially as described in Pai, 2021, High-throughput and single-cell T cell receptor sequencing technologies, Nat methods 18(8):881-892, incorporated by reference. Any suitable sequencing method may be used to determine the sequences of T-cell receptors in the lymph or drain fluid.

T cells express T cell receptors (TCRs) made up of recombined TCRα and TCRβ chains, which mediate recognition of major histocompatibility complex (MHC)-antigen complexes and drive the antigen-specific adaptive immune response in cancer. That phenotypic specification is largely driven by the specific TCR expressed on each T cell's surface. The TCR has two chains, TCRα and TCRβ, made via combinatorial somatic rearrangement of multiple variable (V), diversity (D) (for the β-chain only), joining (J) and constant (C) gene segments. Recombination (along with minor indel activity) generates a TCR repertoire with diversity up to 2×10\hat{ }19 unique TCRαβ pairs. Due to the low probability of somatic recombination making one V(D) J rearrangement twice in an individual, the TCR sequence is thought to represent a unique T cell clone. Measuring TCR diversity over time can reveal patterns of T cell clonal dynamics that correlate with treatment response or other clinically relevant features.

Most approaches to TCR sequencing involve amplifying TCR nucleic acid by a version of multiplex PCR or RNA-Seq. In multiplex PCR, due the diversity of TCR V genes, one pair of primers is not sufficient to capture all TCR transcripts. To address that, one approach uses multiplex PCR reactions with a set of forward primers complementary to all known V genes and a set of reverse primers for either J or C regions, depending on whether the starting material is genomic DNA (gDNA) or complementary DNA (cDNA), respectively. In this approach, gDNA or RNA is isolated from T cells and subjected to multiple rounds of PCR using these primer sets, which may also contain universal primer binding sequences, adaptors, or complements thereof to for compatibility with subsequent sequencing on high-throughput (NGS) sequencing platforms.

Another approach for TCR sequencing is based on “5′ rapid amplification of cDNA ends”, or RACE. In 5′ RACE, RNA is reverse transcribed by using a reverse transcriptase enzyme with terminal transferase activity that adds untemplated C nucleotides to the 3′ end of the cDNA. A template switch oligonucleotide (TSO) containing a complementary poly(G) stretch then anchors to this untemplated region, enabling the reverse transcriptase to switch templates and continue extending the cDNA to the end of the TSO, which includes a common adaptor sequence. As a result, one pair of primers targeting the 5′ adaptor and the constant region is sufficient to amplify all TCR rearrangements. See Picelli, 2014, Full-length RNA-seq from single cells using Smart-seq2, Nat Protoc 9:171-181 and Freeman, 2009, Profiling the T-cell receptor B-chain repertoire by massively parallel sequencing, Genome Res 19:1817-1824, both incorporated by reference. Either approach (multiplex PCR or 5′ RACE) produces amplicons that can be sequenced to obtain sequence data that includes sequences of the TCRs in the lymph or drain fluid.

Each unique TCR is taken as marking a unique clone, so a count of unique TCRs is a measure of unique T cells informing the sample.

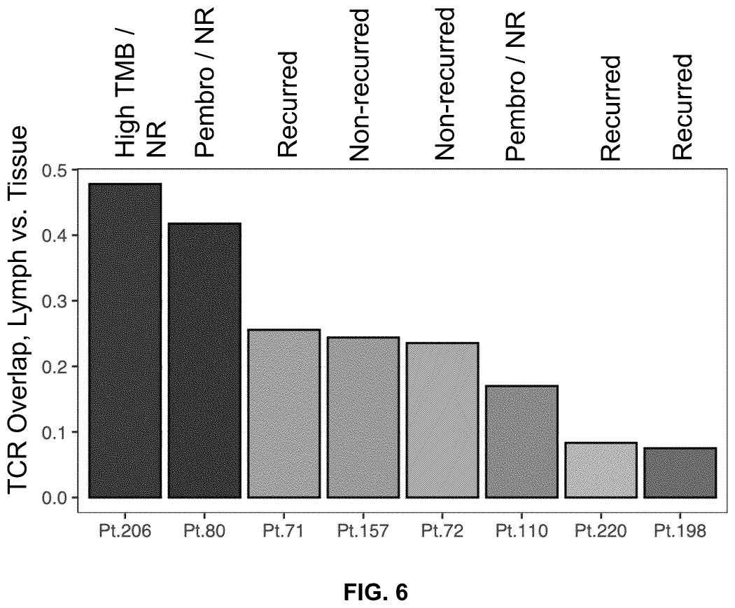

FIG. 6 gives a summary of results from TCR Sequencing. T cell DNA is present in double-spun lymph. As shown, the eight samples from tissue (lymph) have a higher T cell fraction and Simpson clonality than the samples from blood. In these embodiments, the measuring step 115 includes performing amplification from gDNA or RNA from a T-cell from the drain fluid or lymph to produce amplicons and sequencing the amplicons to determine a T-cell receptor (TCR) repertoire of the T-cell. The method may further include providing a report with a measure of clonality of the tumor based on T-cell diversity measured by TCR sequencing.

In some embodiments, the measuring step includes TCR sequencing and correlating TCR diversity measured from lymph in the drain fluid to TCR diversity measured from tissue and predicting that the patient will be responsive to an immunotherapy when tissue- and lymph-derived TCR diversity are highly correlated. Specifically, when the drain fluid is surgical drain fluid (SDF), the measuring step may include TCR sequencing from the SDF, and optionally reporting a measure of clonality of the tumor based on the TCR sequencing.

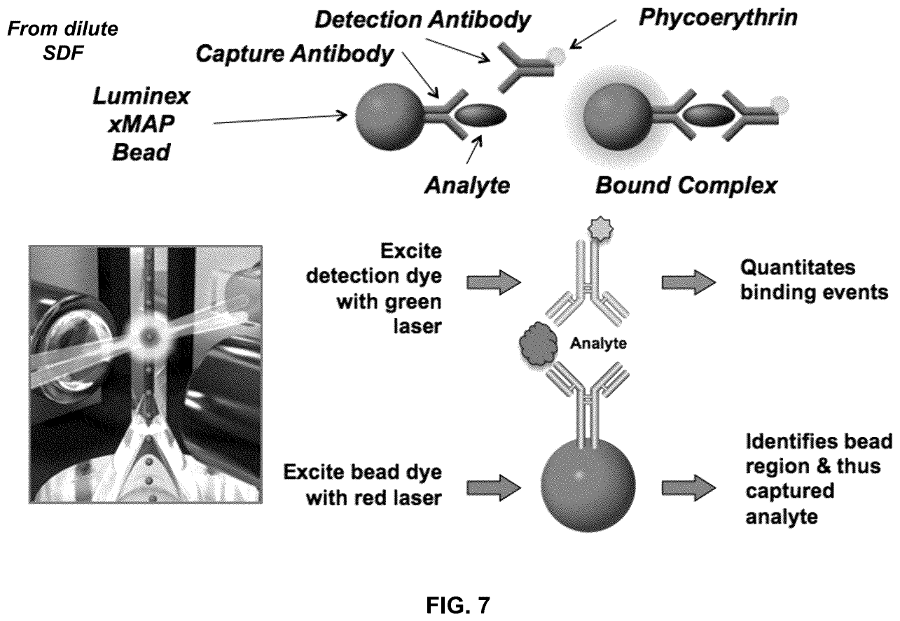

FIG. 7 shows TCR overlap for lymph (drain fluid) versus tissue samples, after controlling for sequencing depth. Other data showed that a number (in a range of hundreds to thousands) of clones were found in both the blood and the tissue sample from each subject. When controlling for sequencing depth by restricting to only the top 1,000 most abundant clones in each sample and identifying shared clones among these subsets.

From TCR Sequencing it is concluded that tumor-associated T cells can be detected in drain fluid. Tumor/SDF clone overlap is high, particularly in inflamed tumors and non-recurred patients. The data indicate that lymph from drain fluid is a rich source of tumor-associated T cells, suggesting non-invasive profiling of the tumor immune microenvironment may be feasible.

### Protein Profiling

Measuring biomarkers in lymph or drain fluid may include looking at or evaluating one or any combination of proteins present in lymph or drain fluid. Protein concentration or presence or abundance of certain pre-determined proteins such as immune proteins in lymph or drain fluid may be measured.

Any assay may be used to detect or measure specific proteins in a sample. For example, proteins may be detected by chromatography (e.g., HPLC); UV absorption (based on strong UV absorption of aromatic side chains specific to amino acids); dye-based methods (involving protein-dye binding and detection of the color change or increase in fluorescence); or other optical methods (e.g., ELISA or lateral flow assays). Certain preferred embodiments have capture proteins using antibodies linked to labeled beads and dyes, specifically sandwiching predetermined proteins between a first detection antibody labelled with a dye (such as phycoerythrin) and a second capture antibody bound to a bead, such as the xMAP bead from Luminex.

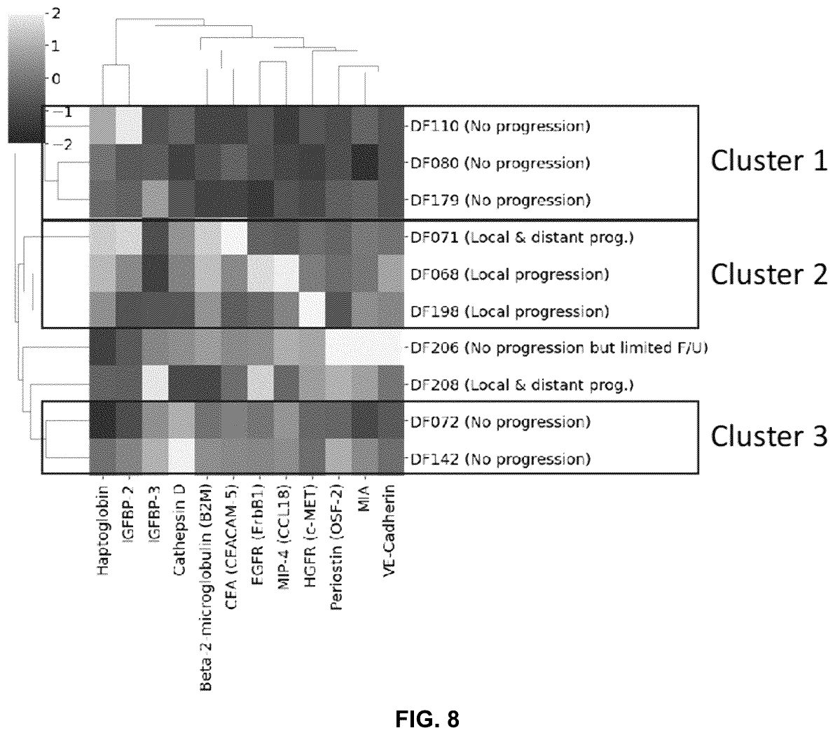

FIG. 8 illustrates multiplex protein quantification using optically labeled antibodies such as those linked to xMAP beads from. Luminex. Samples from HPV− head and neck cancer patients were processed, include recurred, non-recurred, and Pembrolizumab neoadjuvant treated patient. 8-fold dilution series were run in duplicate for two panels: Cell Proliferation and Metastasis 12-Plex and Cytokine/Chemokine/Growth Factor 45-Plex.

The cell proliferation and metastasis panel probed for the beta-2-microglobulin (B2M), cathepsin D, CEA (CEACAM-5), EGFR (ErbB1), haptoglobin, HGFR (c-Met), IGFBP-2, IGFBP-3, MIA, MIP-4 (CCL18), periostin (OSF-2), and VE-cadherin proteins. The cytokine/chemokine/growth factor panel probed for: Th1/Th2: GM-CSF, IFN gamma, IL-1 beta, IL-2, IL-4, IL-5, IL-6, IL-8, IL-12p70, IL-13, IL-18, TNF alpha; Th9/Th17/Th22/Treg: IL-9, IL-10, IL-17A (CTLA-8), IL-21, IL-22, IL-23, IL-27; Inflammatory cytokines: IFN alpha, IL-1 alpha, IL-IRA, IL-7, IL-15, IL-31, TNF beta; Chemokines. Eotaxin (CCL11), GRO alpha (CXCL1), IP-10 (CXCL10), MCP-1 (CCL2), MIP-1 alpha (CCL3), MIP-1 beta (CCL4), RANTES (CCL5), SDF-1 alpha; Growth factors: BDNF, EGF, FGF-2, HGF, NGF beta, PDGF-BB, PIGF-1, SCF, VEGF-A, VEGF-D. Reagents for these panels including antibodies are commercially available from Thermo Fisher Scientific (Waltham, MA).

The panels were performed to detect and quantify proteins from two relevant classes of circulating proteins, to investigate correlations with recurrence, and to look for proteins associated with Pembrolizumab neoadjuvant therapy. In these embodiments, the measuring step 115 includes performing an assay such as an antibody-binding assay to detect a plurality of pre-determined proteins. Such an assay may include binding labeled antibodies to the proteins.

The biomarkers include proteins and preferably include one or more cytokine, chemokine, or growth factor. The proteins may include one or more of: GM-CSF; IFN gamma; IL-1 beta; IL-2; IL-4; IL-5; IL-6; IL-8; IL-12p70; IL-13; IL-18; TNF alpha; Th9/Th17/Th22/Treg: IL-9; IL-10; IL-17A (CTLA-8); IL-21; IL-22; IL-23; IL-27; IFN alpha; IL-1 alpha; IL-IRA; IL-7; IL-15; IL-31; TNF beta; Eotaxin (CCL11); GRO alpha (CXCL1); IP-10 (CXCL10); MCP-1 (CCL2); MIP-1 alpha (CCL3); MIP-1 beta (CCL4); RANTES (CCL5); SDF-1 alpha; BDNF; EGF; FGF-2; HGF; NGF beta; PDGF-BB; PIGF-1; SCF; VEGF-A; VEGF-D; beta-2-microglobulin (B2M); cathepsin D; CEA (CEACAM-5); EGFR (ErbB1); haptoglobin; HGFR (c-Met); IGFBP-2; IGFBP-3; MIA; MIP-4 (CCL18); periostin (OSF-2); and VE-cadherin, ADAM12, AD, ADGRG1, AFP, AGRN, ANGPT2, AOC1, APLN, APOC1, B2M, BDNF, CALR, CCL11, CCL17, CCL18, CCL19, CCL20, CCL23, CCL24, CCL3, CCL4, CCL5, CD244, CD28, CD4, CCL5, CD244, CD28, CD4, CD40-L, CD5, CD8A, CDF15, CEA, CEMIP, CILP2, CLCA2, COL10A1, COL11A1, CREG2, CRTAM, CSF1, CSF2, CST1, CTHRC1, CTLA4, CTSB, CTSD, CX3C1, CXCL1, CXCL10, CXCL11, CXCL12, CXCL13, CXCL5, CXCL8, CXCL9, DCN, EFNA4, EGF, EGFR, ESM1, F12, FASLG, FCGR3A, FGF2, GAL1, GAL9, GGH, GM-CSF, GRN, GZMA, GZMB, GZMH, Haptoglobin, HGF, HO-1, OCOSLG, IFNa, IFNg, IGFBP2, IGFBP3, IGFL1, IGHV4-28, IGLL5, IL-la, IL-1b, IL10, IL11, IL12, IL12P70, IL12RB1, IL13, IL15, 17A, IL18k, ILIRA, IL2, IL21, IL22, IL23, IL27, IL31, IL33, IL4, IL5, IL6, IL7, IL8, IL9, INHBA, KIR3DL1, KLK11, KLK13, KLK14, KLK4, KLK8, KLRD1, LAG3, LAMB3, LAMC2, LAMP3, LY6K, MCP-1, MCP-2, MCP-3, MCP-4, MDK, MET, MIA, MIC-A/B, MMP1, MMP10, MMP11, MMP12, MMP13, MMP2, MMP3, MMP7, MMP9, MUC-16, NCR1, NECTIN1, NECTIN4, NFFB, NOS3, NPC2, NMXPH4, OSF2, PIGF1, PAEP, PDL1, PDL2, PDCD1, PDGFB, PGF, PLA2G2D, PLAC1, PLAU, POSTN, PTHLH, PTN, SCF, SCG5, SDC4, SDFIA, SLIT1, SPP1, STC2, TGFA, TNF, TNFA, TNFB, TNRSF12A, TNFRSF21, TNFRSF4, TNFRSF9, TNFSF14, TRAIL, TREM2, TWEAK, ULBP2, VE-cahedrin, VEGFA, VEGFD, VEGFR-2, WNT2, and ZP3. In preferred embodiments, a concentration of one more IL-1 beta, IL6, FGF-2, and IL-7 are measured in lymph or drain fluid, and MRD, recurrence, or metastasis is reported or predicted when the measured concentration(s) meet or exceed a threshold.

The measured protein levels can be presented in any suitable format including, for example, as a heat map, which revels that know outcomes (e.g., cancer progression versus no progression) can be correlated to protein abundances in the sample, showing certain protein abundances to be predictive of the presence, future progression of, or metastatic potential of a tumor in the subject.

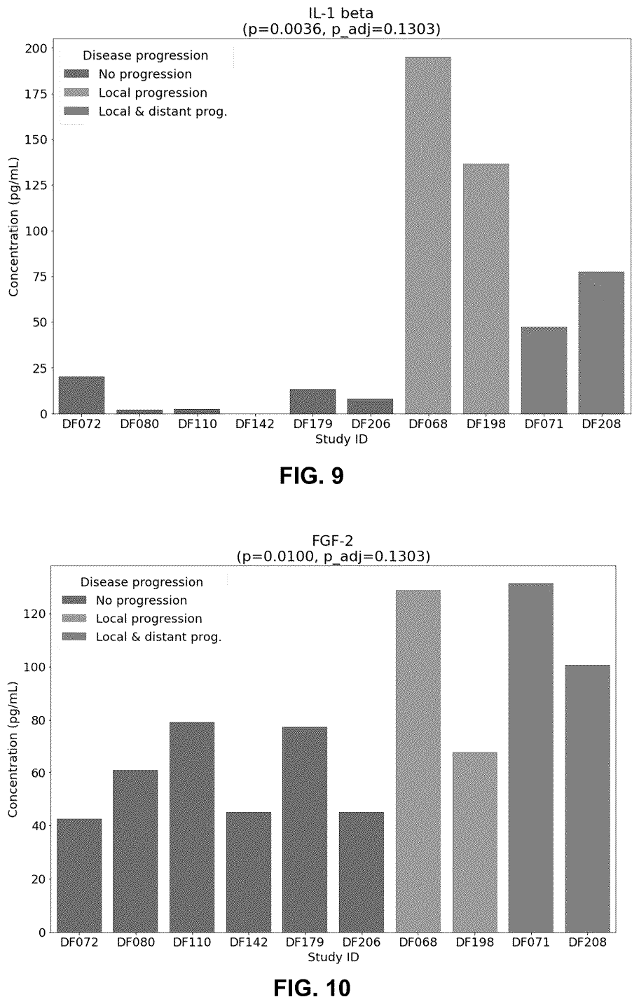

FIG. 9 is a heat map of proteins from a metastasis panel. The heat map presents evidence of clustering by recurrence. Cluster 1 shows no progression in patients with low concentrations across most analytes. Intriguingly, Cluster 1 contains the two patients on neoadjuvant therapy (110 & 080). This suggests that that lymph or drain fluid (which includes lymph) contains biomarkers that not only are predictive of tumor aggressiveness but also show therapeutic efficacy and aid in therapeutic selection. In the heat map, Cluster 2 shows patients with tumors that exhibited progression. Where the tumor exhibited progression, the drain fluid included relatively high levels of haptoglobin, IGFBP-2, and B2M. Cluster 3 includes patients with tumors that exhibited no progression and for whom drain fluid had relatively low levels of biomarkers such as haptoglobin, but higher levels of Cathepsin D.

Methods of the invention may be used to probe for levels of certain proteins and, where those have been correlated to known outcomes, use those proteins in drain fluid as predictive of future tumor aggressiveness in a patient. Results have indicated that cytokines such as IL-1β, FGF-2, IL-6, and IL-7 in drain fluid are potentially valuable and informative biomarkers of tumor aggressiveness.

FIG. 10 shows concentration of IL-1β in drain fluid for patients with known outcomes. As can be seen for patients DF068, DF198, DF071, and DF208, IL-1β is significantly predictive of recurrence. It is understood that IL-1β promotes migration and invasion by cancer cells, triggers an aggressive cancer phenotype, drives immunosuppression, and induces local tumor development and angiogenesis.

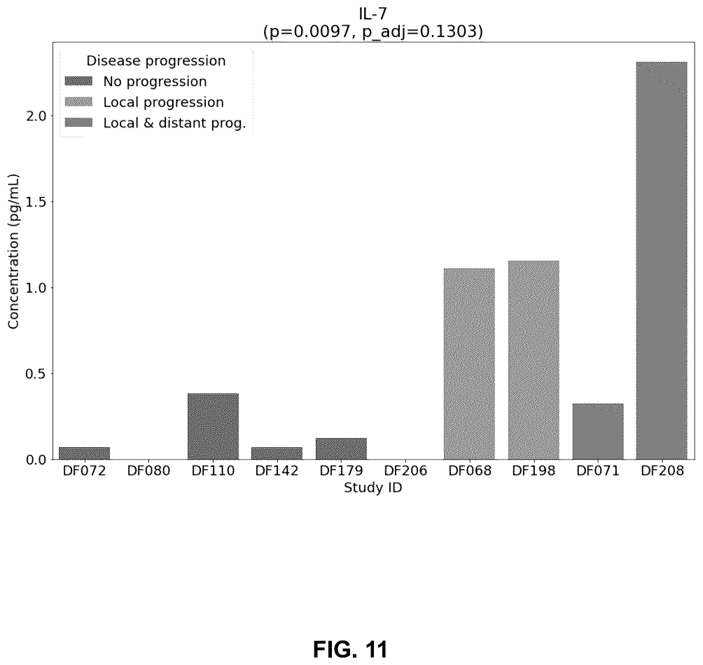

FIG. 11 shows that concentration of FGF-2 in drain fluid is significantly predictive of recurrence. Dysregulated FGF/FGFR signaling is associated with aggressive cancer phenotypes, enhanced chemotherapy resistance and poor clinical outcomes.

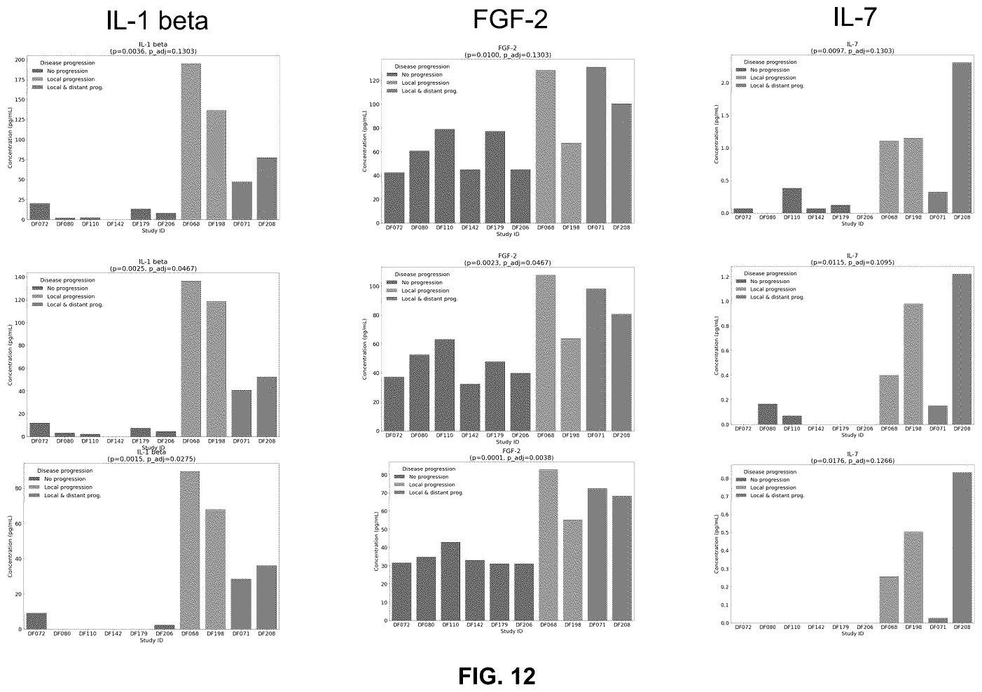

FIG. 12 shows that concentration of IL-7 in drain fluid is significantly predictive of recurrence. The IL-7 protein contributes to the invasiveness of cancer cells by promoting Epithelial-Mesenchymal Transition (EMT).

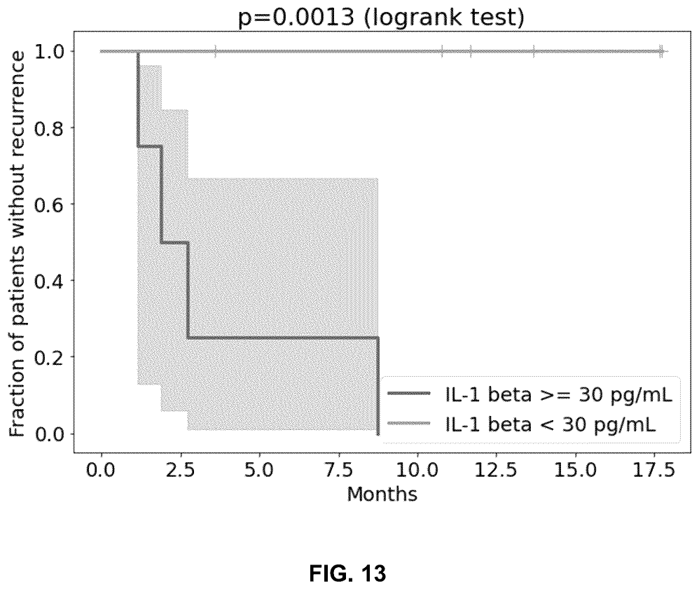

FIG. 13 shows that IL-1 beta, FGF-2 & IL-7 effects are robust across dilutions. These data suggest that cytokines such as IL-1 beta, FGF-2 & IL-7 can be measured in drain fluid and are predictive of cancer recurrence, e.g., after treatment. In fact, levels of those cytokines in drain fluid can be used to, and has shown effective to, stratify patients by recurrence over time after treatment.

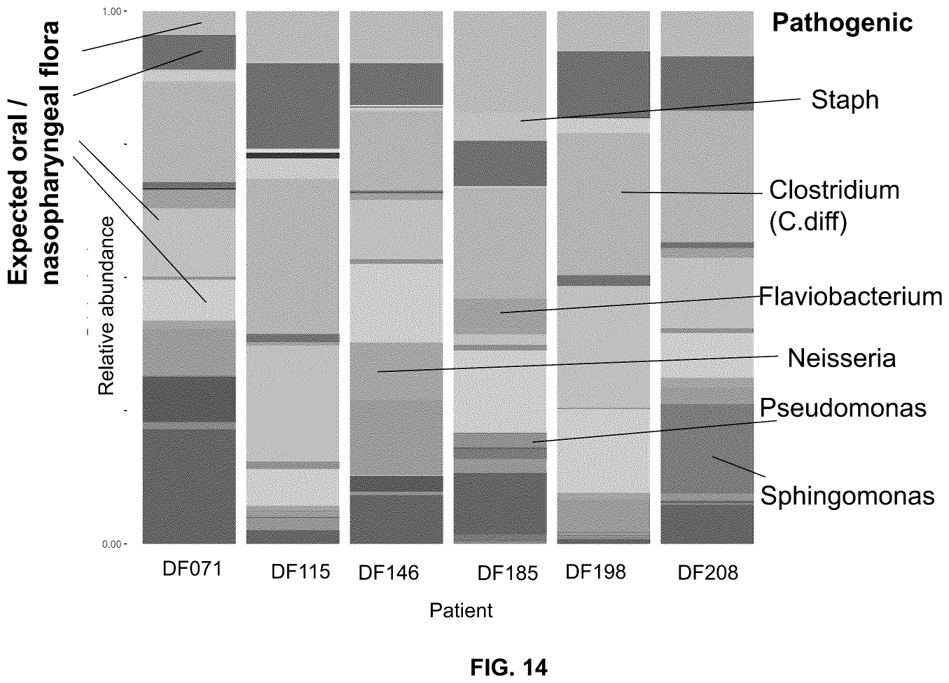

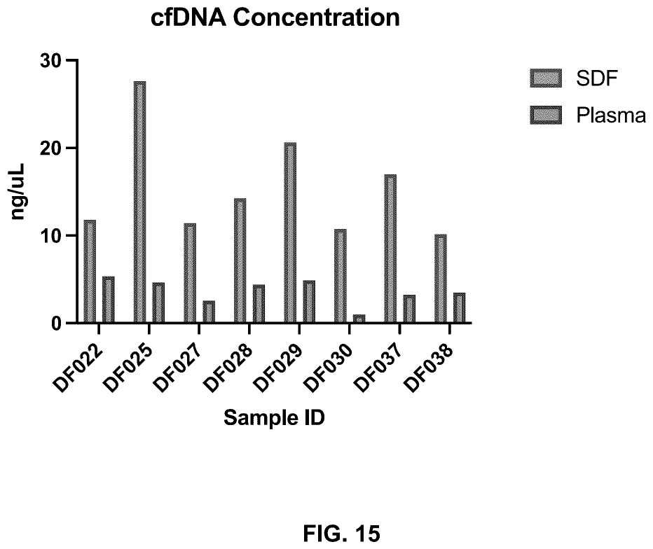

FIG. 14 shows that IL-1β stratifies recurrence. Choosing 30 pg/mL as the cutoff, IL-1β fully stratifies recurrence in the 10 patients of the study. The results presented here support the conclusion that immune- and metastasis-related proteins can be detected in lymph and drain fluid. Here, 52 proteins were detected in at least one patient at one or more dilution. Significantly, IL-1β, FGF-2, IL-6, IL-7, and other biologically relevant proteins show large effects predicting cancer recurrence. The study demonstrates that lymph from drain fluid is a rich source of immune and metastasis-related protein, and that these proteins have utility is predicting metastasis and disease aggressiveness. Among other things, high levels one or more of haptoglobin, IGFBP-2, and B2M in lymph or drain fluid predicts metastasis or recurrence of treatment. Similarly, high levels of one or more of FGF-2, IL-1 beta, IL-6, and IL-7 in lymph or drain fluid is predictive of significant progression of the tumor or recurrence of the tumor after treatment. Method of the invention (e.g., measuring protein concentration in lymph or drain fluid) may be performed after a patient has been treated to eradicate the tumor (e.g., to measure IL-1 beta) and predicting recurrence of the tumor when that marker protein is above a threshold level (e.g., when IL-1 beta is present at a concentration of at least 30 pg/mL in the lymph or drain fluid).

### Microbial Profiling

Methods of the invention are introduced and discussed generally in terms of predicting tumor aggressiveness (e.g., which may include detecting minimal residual disease (MRD), predicting recurrence, or predicting metastasis). A discovery of the methods of the invention is that biomarkers in lymph or drain fluid are predictive of bacterial infection and sepsis earlier than other assays for detecting sepsis. Specifically, the lymph or drain fluid may be probed for microbial markers. When those markers are detected and are specific for sepsis-associated or pathogenic bacteria, the results correlate to the development of sepsis. In certain preferred embodiments, the microbial biomarkers are probed at two different times (e.g., at least 8 hours apart, preferably 12 hours or a day part), When a pathogenic bacterium-specific biomarker is increasing in concentration across those two times, the result is predictive of sepsis. The method has been performed by specifically sequencing gene segments for the bacterial 16S ribosomal rRNA molecule.

In certain microbial detection embodiments, the method includes measuring a level of a microbial biomarker in the drain fluid. In the microbial detection embodiments, the measuring may include sequencing microbial ribosomal nucleic acid and identifying a genus of a bacterium. Sequencing those gene segments may reveal a plurality of different bacteria in the body, without the requirement of isolating and culturing the individual organisms. In that sense, the invention provides for metagenomics by 16s rRNA sequencing.

Microbial metagenomics for sepsis detection was performed for eight patients with HPV-head and neck cancer. That is, drain fluid was obtained from a surgical site, i.e., surgical drain fluid (SDF). Gene segments for 16S RNA was sequenced from that drain fluid and mapped to reference data (e.g., Genbank) to identify at least a genus of each distinct 16S segment sequenced. SSI outcomes for the patients were known.

The microbial 16S sequencing was performed to identify presence and spectrum of bacteria in lymph from surgical drains. The 16S gene was a useful first place to start, but any conserved gene could be used. The skilled artisan will appreciate that a variety of genes could be used. The methods are useful to assess presence of potential pathogenic species.

In the microbial detection embodiments, the method may include measuring the microbial biomarker at two different time and detecting an increasing level of a microbe in the patient. In the microbial detection embodiments, the method may include reporting or predicting sepsis in the patient when the increasing level of the microbe is present, e.g., particularly when a genus of the microbe is Staphylococcus, Clostridium, Flaviobacterium, Neisseria, Pseudomonas, or Sphingomonas.

The method was performed for several patients and the relative abundances of a plurality of bacteria are graphed.

FIG. 12 is a graph showing relative abundances of plurality of bacteria in drain fluid identified by 16s sequencing. For clarity, the normal, healthy flora (the “expected oral/nasopharyngeal” bacteria) are not specifically labelled. The segments of the graph showing an abundance of certain pathogenic bacteria (of the Genera Staphylococcus, Clostridium, Flaviobacterium, Neisseria, Pseudomonas, and Sphingomonas) are labelled.

Those data suggest that bacterial DNA can be detected in drain fluid. The expected flora for neck surgery can be detected in all patients. Importantly, potentially pathogenic strains are observed in several patients. This study demonstrates that lymph from drain fluid is abundant in both commensal and pathogenic bacterial species, suggesting that drain fluid may be used for monitoring for surgical site infection.

### DNA Characterization

An insight of the disclosure is that lymph, such as is found in drain fluid including surgical drain fluid as well as wound drain fluid, is a source of cell-free DNA (cfDNA). The disclosure includes the insight that cfDNA in surgical drain fluid (SDF) exhibits SDF-specific characteristic patterns of abundance and fragmentation. For this, the abundance of cfDNA was measured in SDF for a number of patients.

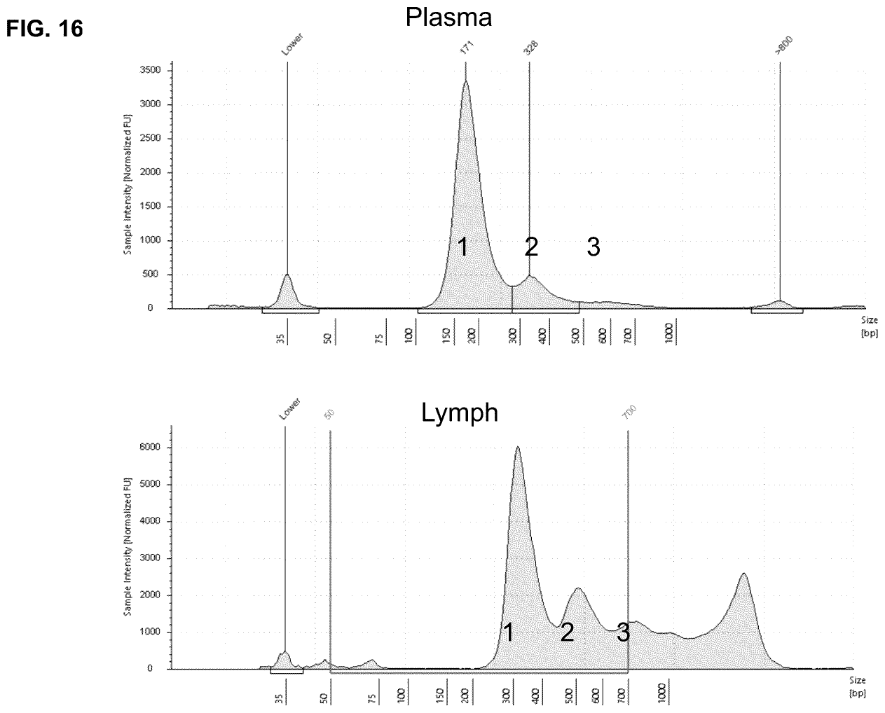

FIG. 16 shows that the yield of cfDNA from lymph in SDF is substantially higher than from plasma. One notable feature is the different, approaching orders of magnitude in difference, concentration of cfDNA ([cfDNA]), here presented in ng/μL, in SDF versus plasma. It is understood that cfDNA is a rich source of clinically important information and also that tumors shed abundant cfDNA. It has previously been theorized that tumors shed cfDNA into the blood stream. Here, it may be theorized that many tumors including, for example, head and neck tumors, colorectal tumors, mammary tumors, and various other ones, may initial shed cfDNA into the lymphatic system (which ultimately feeds into the arteriovenous circulatory system at the venous angle). However, surgical drain fluid includes lymph with such material more proximal to such tumors that the bloodstream, so material from such tumors may be more abundant in lymph than in blood. The figure provides evidence that this is the case. Results indicate that lymph extracted DNA concentrations are 4.8±2.8-fold higher than from blood or plasma.

A consequence of this insight is that techniques or methods that rely on cfDNA in blood or plasma, especially when performed for information about a tumor, may provide more information, at a better resolution, and more readily when the sample is lymph such as in drain fluid including surgical drain fluid (or wound drain fluid).

Not only is cfDNA more abundant in lymph and drain fluid than in plasma, the cfDNA in lymph and drain fluid is characterized by a distinctive fragmentation pattern.

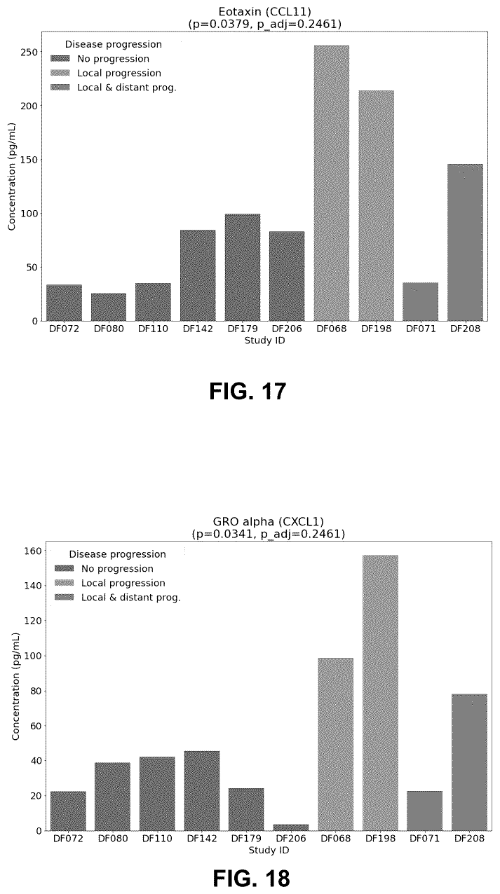

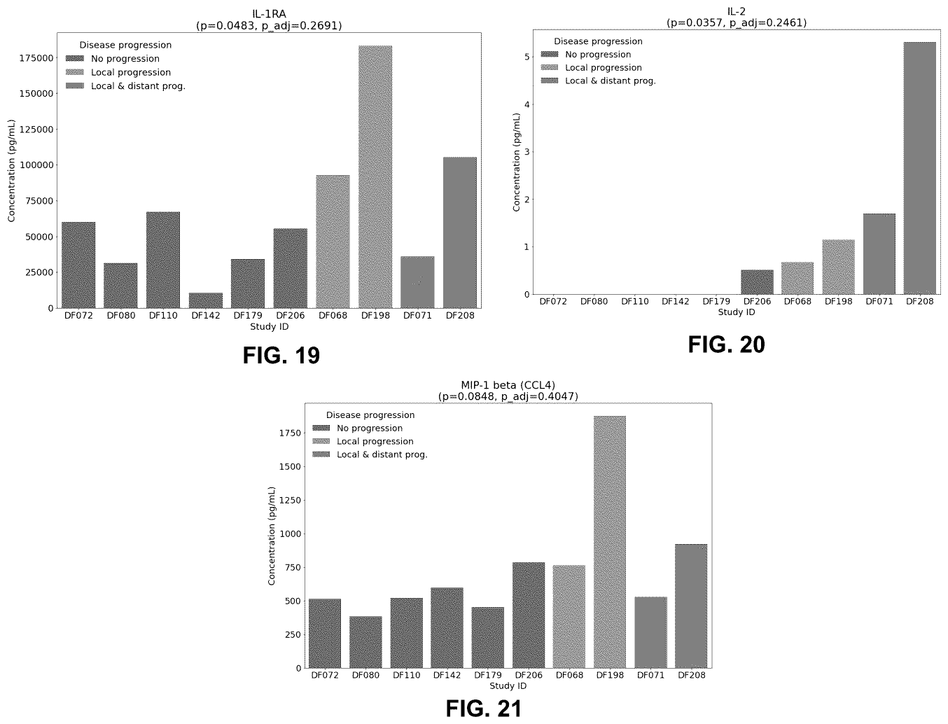

FIG. 17 shows that that the fragmentation patterns of cfDNA found in lymph is distinct from that found in plasma. Specifically, as known, cfDNA in blood or plasma is characterized by a distinct and prominent peak at about 171 bp in length. In contrast, cfDNA from lymph or drain fluid is characterized by numerous peaks (8 visible to inspection) several quite distinct and prominent compared to that from plasma. In particular, the fragmentation pattern from lymph, compared to plasma, has more prominent peaks at about 500 bp, 700 bp, and 1,250 bp in length. Those reliably available larger peaks offer rich source of tumor DNA useful for studying and predicting the aggressiveness of a tumor.

Without being bound by any mechanism, it may be theorized that DNA is packaged in histones, and histone associated DNA is protected from degradation by nucleases. Cell-free DNA from plasma is typically cleaved between nearly all histones. In contrast, lymph cell-free DNA has different nuclease activity than plasma, resulting in the notable peaks at larger fragment sizes.

### CONCLUSIONS

Results are presented here from measuring a plurality of distinct biomarkers in lymph or drain fluid, in particular from surgical drain fluid, fluid that drains from or is irrigated from, a site of surgery. Embodiments herein show that biomarkers measured in drain fluid from the site of a tumor resection, or a lymphadenectomy are predictive of tumor aggressiveness. The information from such biomarkers may be used by a clinician to inform the approach to treating a disease.

Methods of the invention are useful for (i) MRD Detection, (ii) characterizing tumor immunology, (iii) early detection of surgical site infection (SSI), and (iv) the prediction of metastasis. For (i) MRD Detection, data show that mutations in lymph predict cancer recurrence in HPV− H&N cancer. It is shown that the fraction of tumor DNA (and mutations) is higher in lymph than plasma. In (ii) tumor immunology, it shown that T cells can be profiled in lymph from surgical drains. Biomarkers measured in lymph or drain fluid is useful to characterize the tumor immune microenvironment. For detecting (iii) surgical site infection, results identify that lymph or drain fluid is useful to identify infection prior to sepsis detection by other methods. Further, (iv) metastasis may be predicted by measuring any of a number of biomarkers in lymph or drain fluid. Methods of the invention are useful to discover & predict disease trajectory and specifically to profile and characterize the aggressiveness of tumor. In particular, methods of the invention are useful after a tumor resection to monitor biomarkers in lymph or drain fluid (e.g., that drains from the surgical site of tumor resection) for biomarkers that indicate MRD or are predictive of recurrence.

### EXAMPLES

### Example 1: Protein Profiling

Analysis was performed to characterize the protein makeup of surgical drain fluid (SDF). The SDF characterization included drain fluid from 10 patients analyzed on the Luminex platform. The Luminex measures fluorescence (MFI) using a bead-linked capture antibody and a fluorophore-linked reporter antibody, both specific to a protein of interest. MFI values are converted to pg/mL using standards. For this study, 2 separate assays were performed: 12-plex and 45-plex. Each patient sample was analyzed across 8 dilutions, with 2 technical replicates each. Standards serve as a useful quality check for each analyte.

Known quantity (pg/mL) of analyte were prepared, instrument response was measured (MFI), and a curve was fitted to convert MFI to pg/ML. Doing so allowed the conversion of instrument responses for samples (the “unknowns”, i.e., the patient samples) to comparable quantities in units of pg/mL. Because true concentrations are known for standards, the quality of each run can be assessed. Results indicate a successful run for both the 12- and 45-plex. Results show evidence of clustering by recurrence.

FIG. 9 provides a heatmap from the 12-plex assay showing results from the protein profiling. The heatmap shows normalized concentrations (z-scores) clustered across both analytes and patients. Cluster 1 (no progression): patients with low concentrations across most analytes. Intriguingly, this cluster contains the two patients on neoadjuvant therapy (110 & 080). Cluster 2 (progression): high haptoglobin, IGFBP-2 & B2M. Cluster 3 (no progression): low haptoglobin, high Cathepsin D.

FIG. 10, FIG. 11, and FIG. 12 reveal, for the 45-plex: IL-1 beta, FGF-2, IL-6, and IL-7 show increased protein concentrations in the 4 recurred patients. FGF-2, IL-1 beta, IL-6, and IL-7 show large effects predicting recurrence and are nominally significant before correction.

FIG. 13 shows that for IL-1 beta, FGF-2 & IL-7 effects are robust across dilutions.

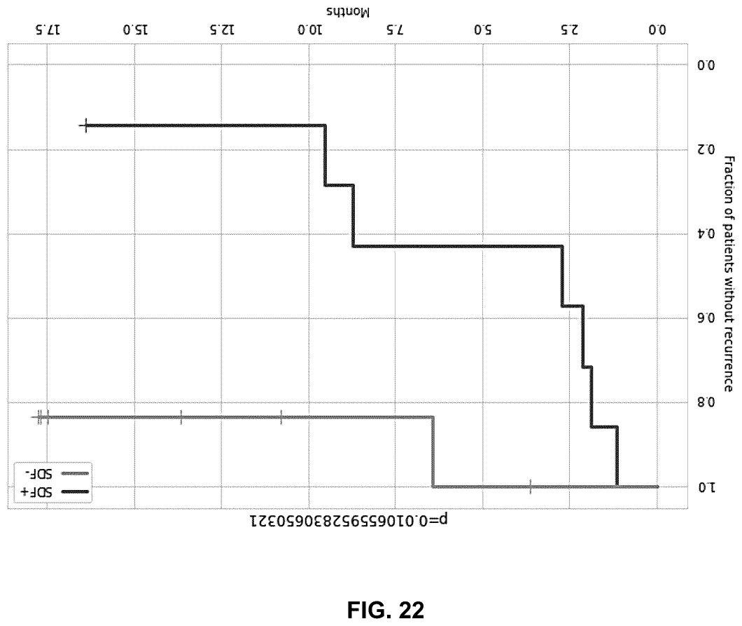

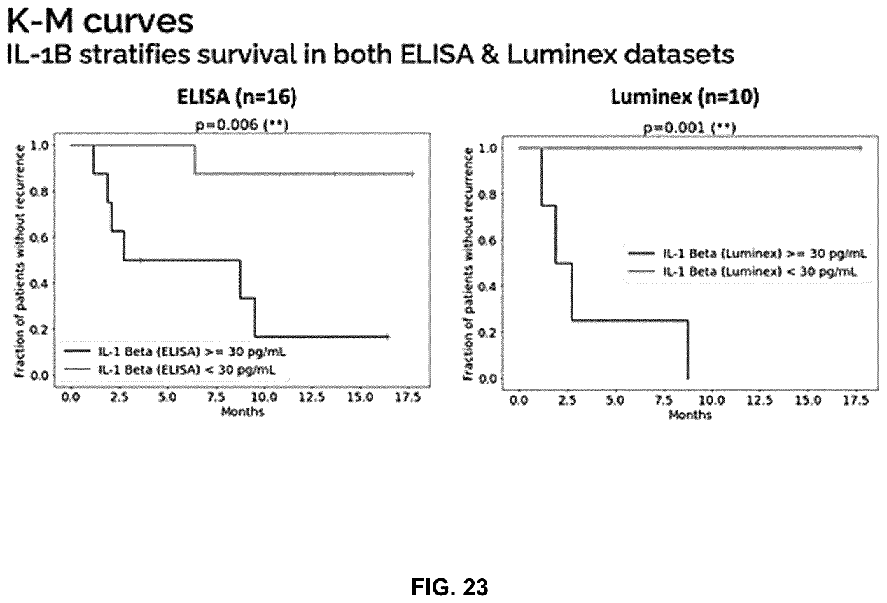

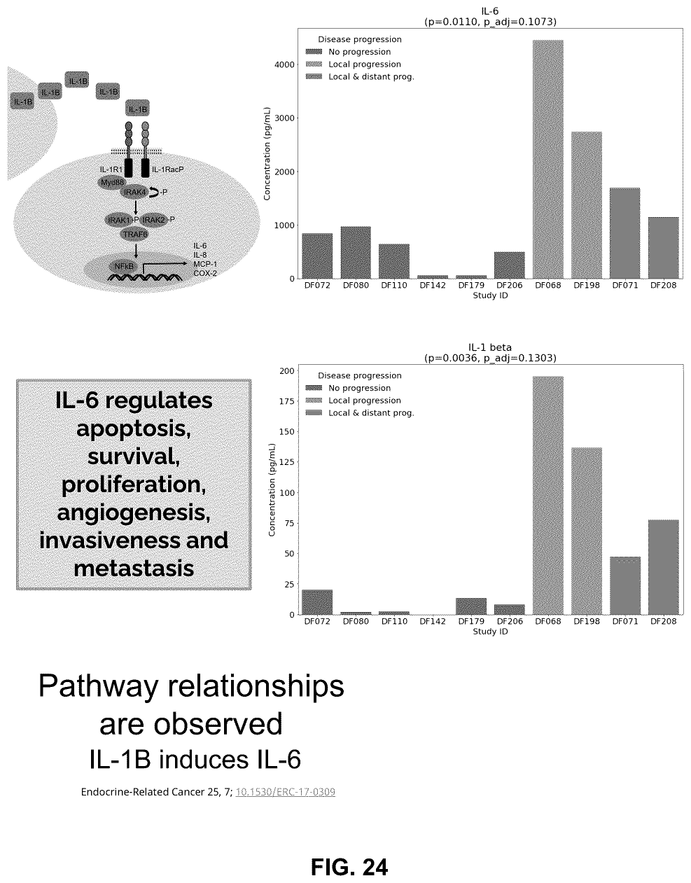

FIG. 21, FIG. 22, FIG. 23, and FIG. 24 also indicate that eotaxin, GRO alpha (CXCL1), IL-IRA, IL-2, and MIP-1 beta (CCL4) at high concentrations may correlate with progression. These results support the conclusion that a variety of proteins (e.g., cytokines, chemokines, growth factors, metastasis-implicated proteins) in lymph or drain fluid may correlate to prediction of future tumor progression. In one specific example,

FIG. 14 shows that IL-1 beta in SDF is a predictor of survival. Choosing 30 pg/mL as the cutoff, IL-1 perfectly stratifies recurrence in our 10 patients.

The presented data support the conclusion that it is possible to measure tumor-predictive proteins in SDF (e.g., in both the 12-plex & 45-plex panels) via an assay with labeled antibodies such as an ELISA or lateral flow assay or the Luminex system. The 12-plex results show clinically relevant clustering with 2 clusters of no disease progression and 1 cluster with recurred patients. Interestingly, patients on neoadjuvant therapy (80 & 110) cluster close together. Some 45-plex analytes are significant individually even after multiple testing correction. Here, IL-1 beta, FGF-2 & IL-7 are significant at 1:4 and 1:8 dilutions, and IL-6 is significant at 1:256 dilution.

### Example 2: Proteomic Study (Lymph v Plasma)

A proteomic study of lymph and plasma was conducted. Protein profiling for lymph fluid and plasma specimens from a cohort of 44 HPV negative head and neck cancer patients was carried out using a proximity extension assay. The assay included lymph and plasma sample collection and preparation from 44 HPV negative head and neck cancer patients. The assay used matched antibody pairs where each antibody is bound to a unique oligonucleotide-barcode. After sample preparation, matched pairs of antibodies for each target protein in lymph and plasma sample were added to the sample solution. When each antibody binds to the target protein in sample solution, and is in close proximity with the other, the oligonucleotide-barcode sequence hybridizes. The barcode sequence was then amplified using known techniques in the art, for example, DNA polymerase. Once amplified, the amplicons are detected and measured using techniques including but not limited to qPCR and NGS.

### Experimental Protocol:

Sample Diluent was thawed, vortexed, and emptied into a multichannel pipette reservoir to have a minimum volume of 15 mL. 96-well plates are labeled 1:2 and 1:10 dilution plate. 5 μL of the Sample Diluent is transferred to each well of columns 1-11 and positions A-B in column 12 on the 1:2 96-well plate using reverse pipetting. 9 μL of Sample Diluent is transferred to each well of columns 1-11 and positions A-B in column 12 on the 1:10 96-well plate, using reverse pipetting. The sample plate was then vortexed using MixMate® at 2000 for 30 seconds and the liquid was spun down at 400-1000×g, for 1 minute at room temperature. 5 μL of the samples and sample controls were transferred to the 96 well plate labeled 1:2 using forward pipetting. 1 μL of samples and sample controls were transferred to the 96 well plate labeled 1:10 dilution using forward pipetting. Original sample plate and dilution plates were sealed with adhesive plastic film. Dilution plates were vortexed using MixMate® at 2000 rpm for 30 seconds. The content was spun down at 400-1000×g, for 1 minute at room temperature. Wells were double checked for same volume and deviations were noted.

An Incubation mix was prepared according to the following table:

| Incubation mix | per 96-well plate (μL) |
|---|---|
| Olink ® Target 96 Incubation Solution | 280 |
| Olink ® Target 96 Incubation Stabilizer | 40 |
| Olink ® Target 96 A-probes | 40 |
| Olink ® Target 96 B-probes | 40 |
| TOTAL | 400 |

The incubation mix was vortexed and spin down. 47 μL of Incubation mix was added to each well of an 8-well strip. 3 μL of Incubation mix was transferred to each well of a 96-well plate labeled “Incubation Plate” by reverse pipetting. 1 μL of each sample was added to the Incubation Plate using a multi-channel pipette to the bottom of the well. In column 12, 1 μL of Negative Control was added to three wells, 1 μL of Interplate Control was added to three wells and pooled plasma Sample Control was added to two wells. The plate was sealed with an adhesive plastic film, spun at 400-1000×g for 1 min at room temperature, and incubated overnight at +4° C. An Extension mix was prepared according to the following table:

| Extension mix | per 96-well plate (μL) |
|---|---|
| High Purity Water | 9385 |
| Olink ® Target 96 PEA Solution | 1100 |
| Olink ® Target 96 PEA Enzyme | 55 |
| Olink ® Target 96 PCR Polymerase | 22 |
| TOTAL | 10562 |

The Incubation plate was brought to room temperature, spun at 400-1000×g for 1 minute. The PCR machine was preheated. The Extension mix was vortexed and poured into multichannel pipette reservoir. A timer for 5 minutes was started and 96 μL of Extension mix was transferred to the upper parts of the well walls of the Incubation plate. The plate was then sealed with an adhesive film, vortexed at 2000 rpm for 30 seconds using MixMate® and spun down at 400-1000×g for 1 minute. The Incubation plate was then placed in the thermal cycler and the PEA program was started (50° ° C. 20 min, 95° C. 5 min (95° C. 30s, 54° C. 1 min, 60° C. 1 min)×17, 10° C. hold).

An Olink® 96.96 Integrated Fluidic Circuit (IFC) for Protein Expression was prepared and primed. One control line fluid syringe was injected into each accumulator on the chip, and then the IFC was primed in the Olink Signature Q100 instrument. The Primer Plate was thawed, vortexed, and spun. A Detection mix was prepared according to the following table:

| Detection mix | per 96-well plate (μL) |
|---|---|
| Olink ® Target 96 Detection Solution | 550 |
| High Purity Water | 230 |
| Olink ® Target 96 Detection Enzyme | 7.8 |
| Olink ® Target 96 PCR Polymerase | 3.1 |
| TOTAL | 790.9 |

The Detection mix was vortexed, spun, and 95 μL was added to each well on an 8-well strip. 7.2 μL of the Detection mix was added to each well of a new 96-well plate by reverse pipetting. The new 96-well plate was labelled Sample Plate. The Incubation Plate was then removed from the thermal cycler, spun down, and 2.8 μL of the content was transferred from each well to the corresponding well on the Sample Plate, using forward pipetting. The plate was sealed with an adhesive plastic film, vortex and spun at 400-1000×g, 1 min at room temperature. 5 μL from each well of the Primer Plate and 5 μL of the Sample Plate was transferred into the primed 96.96 IFC left and right inlets, respectively. Bubbles were removed and the chip was loaded in Olink Signature Q100. The plate was run on the Olink Signature Q100.

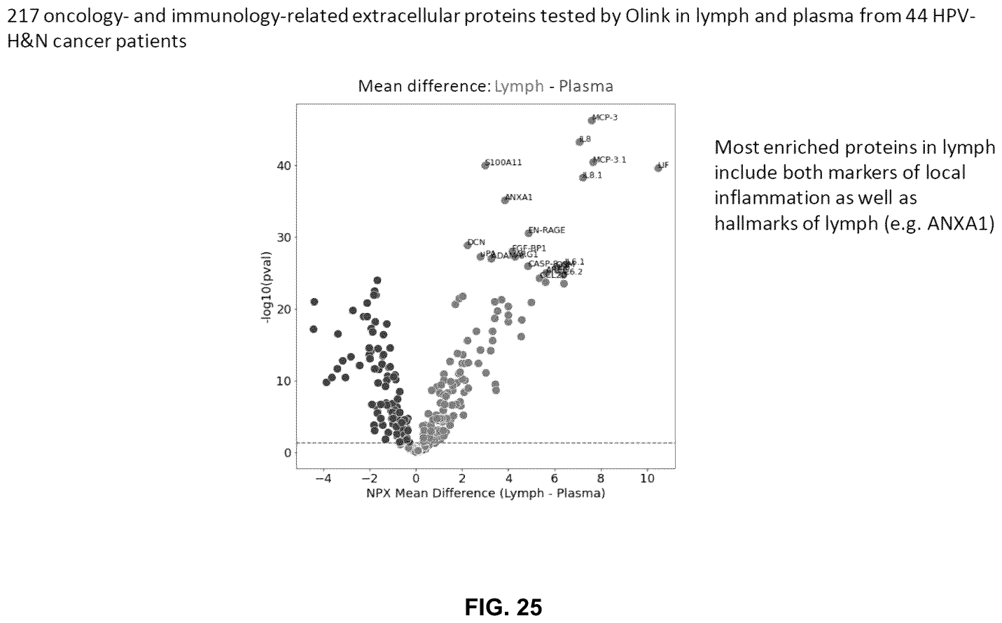

FIG. 25 shows results from protein profiling for lymph and plasma samples. Results show that enriched proteins in lymph include both markers of local inflammation as well as markers that are hallmarks of lymph (for e.g., ANXA 1).

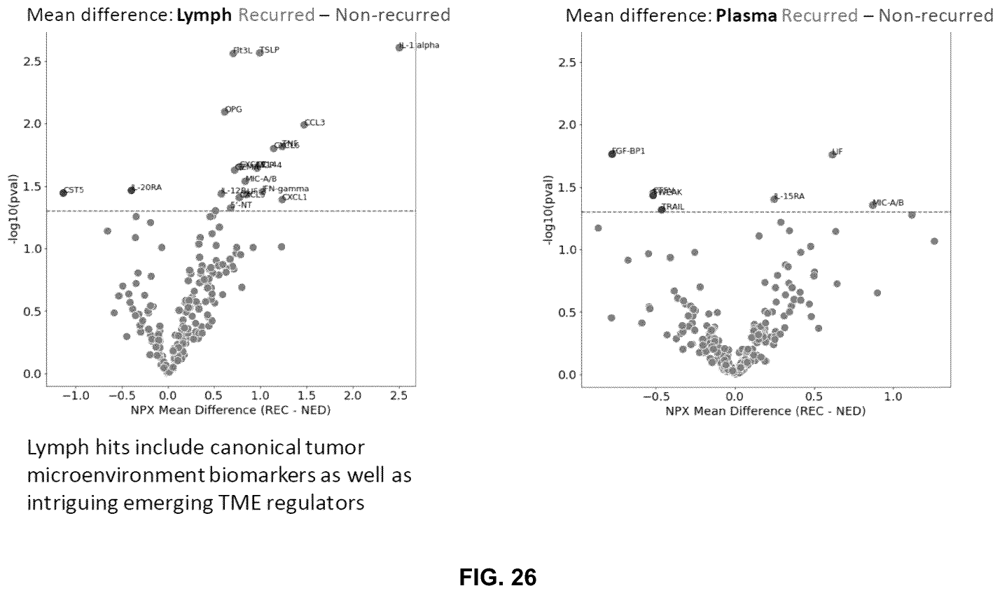

FIG. 26 shows that the lymph is enriched for TME biomarkers, such as TNF and IFN-γ, as well as emerging TME regulators in patients with recurrence of HPV negative head and neck cancer. Such TME-associated proteins in recurred patients are generally absent in the plasma samples.

From these results, it is clear that lymph contains highly differentiated protein repertoires. Lymph shows superior results with respect to plasma for disease recurrence prediction, with proteins associated with recurrence including canonical TME-associated proteins and novel TME regulators. The biomarkers found in the lymph are rarely found in the plasma samples from the same patients. Further, proteins detected in lymph are largely non-overlapping with plasma indicating that lymph provides a better opportunity for unique biomarker discovery over plasma. Thus, surgical drain fluid provides a unique insight into protein biomarkers useful in diagnostics, prediction of recurrence, therapeutic selection and therapeutic efficacy.

What is claimed is:

1. A method for predicting onset of sepsis, the method comprising the steps of:
obtaining lymphatic fluid;
measuring a level of a microbial biomarker in the lymphatic fluid; and
predicting sepsis onset on the basis of the measured level.

2. The method of claim 1, wherein the measuring step comprises sequencing microbial ribosomal nucleic acid and identifying a bacterial class, order, family, genus, or species.

3. The method of claim 1, further comprising measuring the microbial biomarker at two different times and detecting a change in a level of a microbe in the patient.

4. The method of claim 3, further comprising reporting or predicting sepsis in the patient when the level is increasing.

5. The method of claim 4, further comprising predicting sepsis when a genus of the microbe is Staphylococcus, Clostridium, Flaviobacterium, Neisseria, Psueodomanas, or Sphingomonas.

6. The method of claim 4, wherein the lymphatic fluid is obtained by collecting surgical drain fluid from a site of surgery.

7. The method of claim 4, wherein the lymphatic fluid is obtained by collecting surgical drain fluid from a site of surgery.

8. The method of claim 4, wherein the surgical drain fluid is obtained via a drain positioned to collect the drain fluid from a surgical site where a tumor or lymph node has been removed.

9. The method of claim 3, further comprising the step of culturing the microbe.

10. The method of claim 9, further comprising selecting an antibiotic against the microbe.

11. The method of claim 1, further comprising measuring a level of a tumor biomarker in the lymphatic fluid.

12. The method of claim 11, wherein the tumor biomarker is a nucleic acid.

13. The method of claim 11, further comprising the step of sequencing the nucleic acid.

14. The method of claim 11, wherein the tumor biomarker is a protein.

15. The method of claim 14, further comprising characterizing the protein.

16. A method for detecting surgical site infection, the method comprising the steps of:
obtaining lymphatic fluid from a surgical site;
measuring a level of a microbial biomarker in the lymphatic fluid; and
identifying a surgical site infection on the basis of the measured level.

17. The method of claim 16, wherein the measuring step comprises sequencing microbial ribosomal nucleic acid and identifying a bacterial class, order, family, genus, or species.

18. The method of claim 16, further comprising measuring the microbial biomarker at two different times and detecting a change in a level of a microbe in the patient.

19. The method of claim 16, further comprising the step of culturing a microbe.

20. The method of claim 16, further comprising selecting an antibiotic.
